(19) United States
(12) Patent Application Publication
(10) Pub. No.: US 2024/0148832 A1
(43) Pub. Date: May 9, 2024

(54) Astrocyte Interleukin-3 Reprograms Microglia and Limits Alzheimer`s Disease

(71) Applicant: The General Hospital Corporation, Boston, MA (US)

(72) Inventors: Cameron McAlpine, Boston, MA (US); Filip K. Swirski, Swampscott, MA (US); Rudolph E. Tanzi, Milton, MA (US)

(21) Appl. No.: 18/274,677

(22) PCT Filed: Feb. 4, 2022
(86) PCT No.: PCT/US2022/015238

§ 371 Date: Jul. 27, 2023

Related U.S. Application Data
(60) Provisional application No. 63/146,015, filed on Feb. 5, 2021.

(51) Int. Cl. A61K 38/20; A61K 9/00; A61P 25/28

(52) U.S. Cl. CPC A61K 38/202; A61K 9/0019; A61P 25/28

(57) ABSTRACT

Described herein are compositions and methods targeting IL-3 signaling for reducing Alzheimer's disease (AD)-related pathology.

### CLAIM OF PRIORITY

This application claims the benefit of U.S. Provisional Application Ser. No. 63/146,015, filed on Feb. 5, 2021. The entire contents of the foregoing are incorporated herein by reference.

### FEDERALLY SPONSORED RESEARCH OR DEVELOPMENT

This invention was made with Government support under Grant No. HL135752 awarded by the National Institutes of Health. The Government has certain rights in the invention.

### TECHNICAL FIELD

Described herein are compositions and methods targeting IL-3 signaling for reducing Alzheimer's disease (AD)-related pathology.

### BACKGROUND

Communication within the glial cell ecosystem is essential to neuronal and brain health¹⁻³. The influence of glial cells on β-amyloid (Aβ) and neurofibrillary tau accumulation and clearance in Alzheimer's disease (AD) is poorly understood, despite growing awareness that these are therapeutically important interactions^{4,5}.

### SUMMARY

Provided herein are methods for treating a subject with Alzheimer's disease, the methods comprising administering a therapeutically effective amount of an Interleukin 3 Receptor (IL3R) agonist. Also provided are Interleukin 3 Receptor (IL3R) agonists, e.g., pharmace for use in a method of treating a subject with Alzheimer's disease.

In some embodiments, the IL3R agonist is (i) an IL3 peptide or an IL3R polypeptide; or (ii) a nucleic acid encoding an IL3 peptide or a nucleic acid encoding an IL3R peptide.

In some embodiments, the nucleic acid encoding an IL3 peptide or an IL3R polypeptide comprises mRNA.

In some embodiments, the nucleic acid encoding an IL3 peptide or an IL3R polypeptide is in an expression vector. In some embodiments, the expression vector comprises a nucleic acid encoding an IL3 peptide and a promotor that directs expression of the IL3 peptide in astrocytes, optionally a GFAP or Aldh1l1 promoter. In some embodiments, the expression vector comprises a nucleic acid encoding an IL3R polypeptide and a promoter that directs expression of the IL3R polypeptide in microglia, optionally a CD11b or Iba1 promoter.

In some embodiments, the expression vector is a viral vector. In some embodiments, the viral vector is an adeno-associated virus (AAV) vector. In some embodiments, the AAV is selected from the group consisting of AAV9, AAV-F, AAV1, AAV2, AAV3, AAV4, AAV5, AAV6, AAV7, AAV8, AAV2/1, AAV2/2, AAV2/5, AAV2/6, AAV2/7, AAV2/8, AAVrh10, AAV11, and AAV12.

In some embodiments, the IL3R agonist is administered in or formulated in a microvesicle. In some embodiments, the microvesicle comprises a nucleic acid encoding an IL3 peptide and a promotor that directs expression of the IL3 peptide in astrocytes, optionally GFAP or Aldh1l1, and/or a nucleic acid encoding an IL3R polypeptide and a promoter that directs expression of the IL3R polypeptide in microglia, optionally CD11b or Iba1.

In some embodiments, the IL3R agonist is administered into or formulated to be administered into the CNS via infusion or injection into the cerebrospinal fluid (CSF), intrathecally, or by direct injection or infusion using stereotactic methods.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-F. IL-3 protects against β-amyloid accumulation and cognitive impairment in 5×FAD mice. A. Representative images of Aβ in 5-month-old 5×FAD and Il3^{-/-}5×FAD mice. B. Quantification of Aβ area and Aβ plaque size in the cortex (n=55×FAD mice; n=7 Il3^{-/-}5×FAD mice). C. Levels of tris-buffered saline (TBS)- and formic acid (FA)-soluble Aβ40 and Aβ42 in cortex homogenates of 5×FAD and Il3^{-/-}5×FAD mice (n=7 5×FAD mice for TBS soluble Aβ40 and 42, FA-soluble Aβ42, n=6 5×FAD mice for FA-soluble Aβ40; n=7 Il3^{-/-}5×FAD mice for TBS- and FA-soluble Aβ40, n=9 Il3^{-/-}5×FAD mice for TBS-soluble Aβ42; n=8 Il3^{-/-}5×FAD mice for FA-soluble Aβ42). D. Quantification of time in new arm and entries made into the new arm during Y-maze (n=12 5×FAD mice; n=8 Il3^{-/-}5×FAD mice). Morris water maze testing. E. Time for male mice needed to reach hidden platform plotted across training days (two-way ANOVA for groups p=0.0074) and area under the curve (AUC) of escape latency (n=10 5×FAD mice; n=9 Il3^{-/-}5×FAD mice). F. Time in target zone on probe day (8th day) (n=10 5×FAD mice; n=9 Il3^{-/-}5×FAD mice). Filled circles represent male mice, open circles represent female mice. *p<0.05, **p<0.01, ***p<0.001, two-tailed Mann-Whitney U-tests unless otherwise indicated. Error bars indicate mean±SEM.

FIGS. 2A-M. Microglia become responsive to astrocyte-derived IL-3 in Alzheimer's disease. A. IL-3 in plasma (PL) and CSF of WT and 5×FAD mice at 5 and 12 months (m.) of age (n=4 WT 5 m. plasma; n=5 5×FAD 5 m. plasma; n=14 WT 5 m. CSF; n=6 5×FAD 5 m. CSF; n=6 WT 12 m. CSF; n=4 5×FAD 12 m. CSF). One-way ANOVA, multiple comparisons. B. Flow cytometry analysis of the brain of WT, Il3^{GFPfl/fl}, and Il3^{GFPfl/fl}5×FAD mice. C. Quantification of IL3⁺ (GFP⁺) astrocytes in the brain of Il3^{GFPfl/fl} and Il3^{GFPfl/fl}5×FAD mice (n=4). D. Il3 expression in Aldh1l1-GFP⁺ astrocytes, CD11b⁺CD45^{mid} microglia, and CD45⁻ cells sorted from Aldh1l1^{GFP} mice (n=7). E. Representative images showing co-localization of IL-3-GFP⁺ with GFAP⁺ astrocytes in Il3^{GFPfl/fl}. F. Quantification of IL3⁺ astrocytes showing co-localization of IL-3 with GFP⁺ astrocytes in Aldh1l1^{GFP} mice (n=4). G. Flow cytometry and quantification (H) of IL-3Rα⁺ microglia in WT and 5×FAD mice (n=8 5 m. WT mice; n=12 5 m. 5×FAD mice; n=3 8 m. WT mice; n=4 8 m. 5×FAD mice). One-way ANOVA, multiple comparisons. I. Il3rα in microglia (n=5 5 m. WT mice; n=4 5 m. and 8 m. 5×FAD mice; n=3 8 m. WT mice). One-way ANOVA, multiple comparisons. J. p-STAT5 in ex-vivo microglia. K. Representative images showing co-localization of IL-3Rα with Iba1 in proximity to Aβ in 5×FAD mice. L. Il3rα in microglia of WT, Trem2^{-/-}, 5×FAD, and Trem2^{-/-}5×FAD mice displayed as Log₂ fold change (for 4-month-old mice p=3.45e⁻²⁷ for 5×FAD vs WT, p=3.4e⁻⁰⁶ for Trem2^{-/-}5×FAD vs WT, p=1.82e⁻⁰⁸ for Trem2^{-/-}5×FAD vs 5×FAD; for 8-month-old mice p=1.12e⁻⁹³ for 5×FAD vs WT, p=1.4e⁻¹³ for Trem2^{-/-}5×FAD vs WT, p=1.6e⁻⁴⁰ for Trem2^{-/-}5×FAD vs 5×FAD; FDR<0.0002; for WT n=13M/14F at 4 m., n=8M/8F at 8 m.; for Trem2^{-/-} n=11M/12F at 4 m., 2M/2F at 8 m.; for 5×FAD n=14M/14F at 4 m., 10M/9F at 8 m.; and for Trem2^{-/-}5×FAD n=11M/11F at 4 m., 9M/8F at 8 m.). ns=not significant. One-way ANOVA. M. IL3Ra⁺ microglia from 5×FAD and Trem2^{-/-}5×FAD mice (n=4). Two-tailed Mann-Whitney U-test. Groups are of evenly mixed sex. *p<0.05, **p<0.01, ***p<0.001. Error bars indicate mean±SEM.

FIGS. 3A-H. IL-3 signaling correlates with disease pathology in the brain of humans with Alzheimer's disease. A. Representative images of the cortex of control subjects (C) or Alzheimer's disease (AD) patients stained for IL-3 and GFAP. B. Measurement of IL-3 levels in cortex tissue homogenates from control and AD patients (n=15 controls, n=23 AD patients, see Table 6 for characteristics). Two-tailed Mann-Whitney U-test. C. Representative images of the cortex of humans with or without Alzheimer's disease (AD) stained for IL-3Rα and Iba1. D. qPCR analysis of fold change in IL3Rα expression in the frontal cortex of control individuals and AD patients (n=28 controls, n=30 AD patients, see Table 6 for characteristics). Two-tailed Mann-Whitney U-test. E. Assessment of brain IL3Rα expression with APOE genotype (n=30 AD patients). One-way ANOVA, multiple comparisons. F. Correlation of mean IL3Rα expression with years (yrs) of disease duration (n=30 AD patients). Pearson's correlation. G. Correlation of IL3Rα expression with formic acid (FA)-soluble Aβ40 and (H) Aβ42 in the frontal cortex of AD patients (n=23 AD patients). Pearson's correlation. *p<0.05, **p<0.01, ***p<0.001. Open circles represent control subjects and red circles represent AD patients. Error bars indicate mean±SEM.

FIGS. 4A-O. IL-3 reprograms microglia promoting motility and clustering of β-amyloid in the murine brain and in a 3D human AD iPS triculture system. A. Enumeration of microglia in WT, 5×FAD, Il3^{-/-}5×FAD mice (n=10 5×FAD mice; n=8 Il3^{-/-}5×FAD mice). B. Multidimentional scaling (MDS) plot of RNAseq data from microglia of 5-month-old 5×FAD and Il3^{-/-}5×FAD mice (n=12, each data point represents 4 pooled mice of 2M/2F). C. Volcano plot indicating differentially regulated genes (FC>1.6, FDR<0.1, p<0.005) D. Heatmap of key differentially regulated genes, except Il3rα which is not significant. E. Pathway analysis. F. Representative images of microglia and skeletal analysis (n=5 5×FAD mice; n=4 Il3^{-/-}5×FAD mice). G. Representative images and quantification of microglia within 25 μm of Aβ plaques (n=5 5×FAD mice; n=7 Il3^{-/-}5×FAD mice). H. Schematic, computed density gradient, and diffusion rate of microglia surrounding Aβ (n=5 5×FAD mice; n=7 Il3^{-/-}5×FAD mice). Two-way ANOVA. I. Representative 3D images of mouse cortex (634×250×634 μm). Groups are of 5-month-old animals of evenly mixed sex. J. Scheme of 3D microfluidic system where human progenitor derived neurons and astrocytes, with or without AD mutations, are plated in the central chamber while human iPS-derived microglia labeled with CellTracker are plated in side chambers. K. Representative images of IL-3 localization to astrocytes and IL-3Rα to microglia. L. Flow cytometry quantification of migrated microglia with or without human rIL-3 (n=3). M. Representative images and quantification of migrated microglia (n=3 per group for astrocytes+neurons; n=4 AD astrocytes+neurons and AD astrocytes+neurons+rIL3). N. CCL2 and CCL4 levels in media (n=3; except n=2 CCL4 astrocytes+neurons). O. Chemokine and cytokine levels (CCL11, CCL26, CCL17, CXCL10, IL-8, CCL22, and CCL13) in the media of the human AD iPS triculture system. (n=3 except n=2 for CCL13 AD astrocytes+neurons). Error bars indicate mean ±SEM. *p<0.05, **p<0.01, ***p<0.001, two-tailed Mann-Whitney U-tests unless otherwise indicated. Error bars indicate mean±SEM.

FIGS. 5A-N. Astrocyte IL-3 or microglia IL-3Rα deletion instigate while IL-3 infusion resolves β-amyloid burden and cognitive decline. A. Representative images and Aβ quantification of brain sections probed for Aβ and DAPI of 5-month-old Il3^{GFPfl/fl}5×FAD and Il3^{GFPfl/fl}Aldh1l1Cre^{Ert2}5×FAD mice injected with tamoxifen (n=6 Il3^{GFPfl/fl}5×FAD mice; n=9 Il3^{GFPfl/fl}Aldh1l1Cre^{Ert2}5×FAD mice). B. Levels of Aβ40 and Aβ42 in cortex homogenates (n=6 Il3^{GFPfl/fl}5×FAD mice; n=9 Il3^{GFPfl/fl}Aldh1l1Cre^{Ert2}5×FAD mice). C. Gene expression in microglia (n=6 Il3^{GFPfl/fl}5×FAD mice; n=9 Il3^{GFPfl/fl}Aldh1l1Cre^{Ert2}5×FAD mice). D. Representative images and quantification of microglia-Aβ co-localization (n=6 Il3^{GFPfl/fl}5×FAD mice; n=9 Il3^{GFPfl/fl}Aldh1l1Cre^{Ert2}5×FAD mice). E. Y-maze time in new arm (n=6 Il3^{GFPfl/fl}5×FAD mice; n=9 Il3^{GFPfl/fl}Aldh1l1Cre^{Ert2}5×FAD mice). F. Representative images and Aβ quantification of brain sections probed for Aβ and DAPI of 5-month-old Il3ra^{fl/fl}5×FAD and Il3ra^{fl/fl}Cx3cr1Cre^{Ert2}5×FAD mice injected with tamoxifen. (n=7). G. Levels of Aβ40 and Aβ42 in cortex homogenates (n=7). H. Representative images and quantification of microglia-Aβ co-localization (n=7). I. Y-maze time in new arm (n=8 Il3ra^{fl/fl}5×FAD mice; n=6 Il3ra^{fl/fl}Cx3cr1Cre^{Ert2}5×FAD). J. Experimental approach and representative images of brain sections probed for Aβ and DAPI from mice receiving brain infusion of PBS or rIL-3. K. Quantification of cortex Aβ (n=6 Il3^{-/-}5×FAD+PBS; n=5 Il3^{-/-}5×FAD+rIL-3). L. Levels of Aβ40 and Aβ42 in cortex homogenates (n=7). M. Representative images and quantification of microglia within 25 μm of Aβ in the cortex (n=6 Il3^{-/-}5×FAD+PBS; n=5 Il3^{-/-}5×FAD+rIL-3). N. Y-maze time in new arm (n=13 Il3^{-/-}5×FAD+PBS; n=10 Il3^{-/-}5×FAD+rIL-3). Filled circles represent male mice, open circles represent female mice. *p<0.05, **p<0.01, ***p<0.001, two-tailed Mann-Whitney U-tests. Error bars indicate mean±SEM.

FIGS. 6A-B. Astrocyte IL-3 and microglia IL-3Rα production. A. Proportion of IL-3Rα⁺ microglia in the brain of WT mice at various ages (n=4). B. Il3rα transcript expression in brain homogenate of WT mice at various ages (n=4). One-way ANOVA. *p<0.05, **p<0.001. Error bars indicate mean±SEM.

FIGS. 7A-D. Il3rα expression in microglia and characterization of IL-3Rα^{hi} and IL-3Rα^{lo} microglia. A. Analysis of Il3ra and other cytokine receptors expressed in resting microglia, DAM stage 1, and DAM stage 2 microglia. Data are published in Keren-Shaul et al. Cell, 2017 B. Gating strategy for IL-3Rα^{hi} and IL-3Rα^{lo} microglia in 5×FAD mice. C. Flow cytometry analysis of IL-3Rα^{hi} and IL-3Rα^{lo} microglia (n=6 except CD11c and CCL2 where n=3). D. mRNA transcript expression in sorted IL-3Rα^{hi} and IL-3Rα^{lo} microglia (n=7 for IL-3Rα^{lo} microglia except for n=6 for Il6, Itgam, Ptprc, Cd36, and Cd68, n=5 for Il10; n=7 for IL-3Rα^{hi} microglia except for n=6 for Il6, Ptprc and Cd68, n=5 for Il10). *p<0.05, **p<0.01, two-tailed Mann-Whitney U-tests. Error bars indicate mean±SEM.

FIGS. 8A-B. rIL-3 delivery to the cortex or periphery of 5×FAD mice and summary figure. A. Recombinant IL-3 or PBS was delivered into the cortex of 5×FAD mice. Three days later microglia localization to Aβ aggregates was assessed (n=7 PBS mice; n=6 rIL-3 mice). Two-tailed Mann-Whitney U-tests. B. Prior to sacrifice Ymaze behavioral testing was performed and time in the new arm was quantified (n=7 PBS mice; n=8 rIL-3 mice). The amount of Aβ in the cortex of mice was quantified by analyzing histological sections (n=6). Groups of mice are of evenly mixed sex. **p<0.01. Error bars indicate mean±SEM.

FIG. 9. Schematic Illustration of Potential Model of IL-3's role in AD. Astrocytes produce IL-3. In response to Aβ, TREM2 signaling increases microglia IL-3Rα, rendering microglia responsive to astrocyte-derived IL-3. IL-3 signaling instigates microglia transcriptional and functional reprogramming leading to a signature of immune regulation, motility, and migration. IL-3-dependent reprogramming promotes clustering of microglia around AP enabling AP clearance and mitigation of AD pathology.

### DETAILED DESCRIPTION

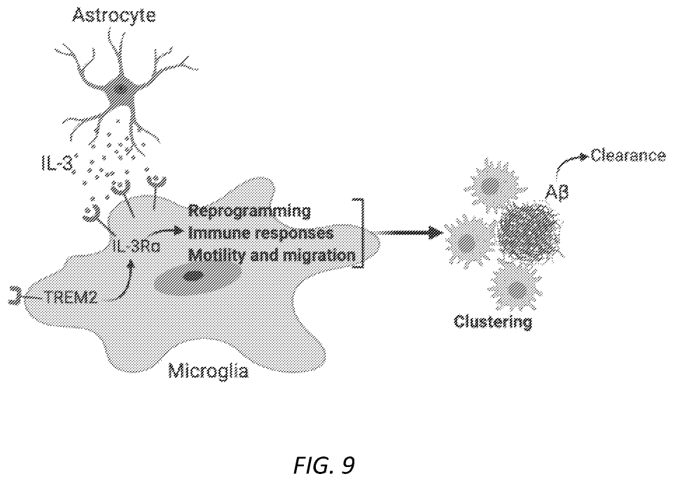

IL-3 is a multifunctional cytokine implicated in inflammatory and autoimmune diseases⁶. In humans, IL-3 levels associate with AD risk⁷⁻¹⁰ and severity^{11,12}, and in vitro studies have implicated IL-3 in neurodegeneration¹³⁻¹⁶. Despite these links, the role of IL-3 in the human or murine AD brain has not been investigated. Without wishing to be bound by theory, as shown herein in humans and mice, astrocyte-sourced interleukin-3 (IL-3) reprograms microglia to ameliorate AD pathology. Upon recognition of Aβ deposits, microglia augment IL-3Rα, IL-3's specific receptor, rendering them responsive to IL-3. Astrocytes constitutively produce IL-3, which elicits transcriptional, morphological, and functional reprograming of microglia endowing them with an acute immune response program, enhanced motility, and the capacity to cluster and clear Aβ and tau aggregates. These changes restrict AD pathology and cognitive decline. Thus, IL-3 is a mediator of astrocyte-microglia crosstalk, microglia programming, and AD pathology (FIG. 9), and IL3 signalling is a target for therapeutic intervention in AD.

### Methods of Treatment

Provided herein are methods for treating neurodegenerative diseases including Alzheimer's Disease (AD); Multiple Sclerosis (MS), e.g., progressive MS; and Amyotrophic Lateral Sclerosis (ALS). The methods described herein can be used to treat or reduce the risk of developing symptoms in subjects with all types of Alzheimer's disease including, but not limited to, familial and sporadic Alzheimer's disease, early onset or late onset Alzheimer's disease. The present methods can be used for any mammalian subject, e.g., human or non-human subjects, e.g., veterinary subjects including primates, cats, dogs, horses, goats, sheep, cows, and so on. The methods include administering a therapeutically effective amount of an IL3R agonist as described herein. The methods can include administering a sufficient dose a plurality of times, e.g., once a week, twice a week, biweekly, monthly, bimonthly, every six weeks, every three months, every four months, every six months, every nine months, or once a year, for a plurality of doses. As used herein, a therapeutically effective amount is an amount to ameliorate one or more symptoms of the disease, e.g., to improve or to stop or slow decline in one or more cognitive, neurological, or motor functions in the subject. Typically, increasing forgetfulness or mild confusion are early symptoms of Alzheimer's disease. Gradually, cognitive impairment associated with Alzheimer's disease leads to memory loss, especially recent memories, disorientation and misinterpreting spatial relationships, difficulty in speaking, writing, thinking, reasoning, changes in personality and behavior resulting in depression, anxiety, social withdrawal, mood swings, distrust in others, irritability and aggressiveness, changes in sleeping habits, wandering, loss of inhibitions, delusions, and eventually death.

The methods can include administering the IL3R agonist systemically, e.g., peripherally via intravenous (IV) or intraperitoneal (IP) infusion or injection; or into the CNS via infusion or injection into the cerebrospinal fluid (CSF), intrathecally, or by direct injection or infusion using stereotactic methods.

### IL3R Agonists

As used herein, IL3R agonists can include one or more of: (i) an IL3 peptide or a nucleic acid encoding an IL3 peptide (e.g., an mRNA); (ii) an IL3R polypeptide or a nucleic acid encoding an IL3R peptide (e.g., an mRNA).

In some embodiments, the IL3 peptide is a human IL3 peptide. An exemplary sequence for human IL3 is provided in GenBank at Acc. No. NP_000579.2, e.g., MSRLPVLLLLQLLVRPGLQAPMTQTTPLKTSWVNCSNMIDEIITHLKQPPLPLLDF NNLNGEDQDILMENNLRRPNLEAFNRAVKSLQNASAIESILKNLLPCLPLATAAP TRHPIHIKDGDWNEFRRKLTFYLKTLENAQAQQTTLSLAIF (SEQ ID NO:1). In some embodiments, a peptide comprising amino acids 20-152 of SEQ ID NO:1 (i.e., lacking the signal sequence) is used. In some embodiments, a peptide comprising amino acids 27-136 of SEQ ID NO:1 is used. In some embodiments, the peptide comprises a sequence that is at least 80%, 85%, 90%, 95%, 97%, or 99% identical to amino acids 1-136, 1-152, 20-136, 20-152, 27-136, or 27-152 of SEQ ID NO:1.

In some embodiments, the agonist comprises daniplestim, which is an engineered IL-3 receptor agonist having the sequence ANCSIMIDEIIHHLKRPPNPLLDPNNLNSEDMDILMERNLRTPNLLAFVRAVKHLE NASGIEAILRNLQPCLPSATAAPSRHPIIIKAGDWQEFREKLTFYLVTLEQAQEQQ (SEQ ID NO:2). See McCubrey et al., Leukemia volume 15, pages 1203-1216 (2001).

In some embodiments, the IL3Ra peptide is a human IL3Ra peptide. An exemplary sequence for human IL3Ra is provided in GenBank at Acc. No. NP_002174.1 (interleukin-3 receptor subunit alpha isoform 1 precursor), or at NP_001254642.1 (interleukin-3 receptor subunit alpha isoform 2 precursor). Variant 2 lacks two in-frame exons in the 5′ coding region as compared to variant 1. The resulting isoform 2 polypeptide lacks an internal segment in the N-terminal region as compared to isoform 1.

An exemplary sequence of human IL3Ra comprises MVLLWLTLLLIALPCLLQTKEDPNPPITNLRMKAKAQQLTWDLNRNVTDIECVK DADYSMPAVNNSYCQFGAISLCEVTNYTVRVANPPFSTWILFPENSGKPWAGAE NLTCWIHDVDFLSCSWAVGPGAPADVQYDLYLNVANRRQQYECLHYKTDAQG TRIGCRFDDISRLSSGSQSSHILVRGRSAAFGIPCTDKFVVFSQIEILTPPNMTAKCN KTHSFMHWKMRSHFNRKFRYELQIQKRMQPVITEQVRDRTSFQLLNPGTYTVQI RARERVYEFLSAWSTPQRFECDQEEGANTRAWRTSLLIALGTLLALVCVFVICRR YLVMQRLFPRIPHMKDPIGDSFQNDKLVVWEAGKAGLEECLVTEVQVVQKT (SEQ ID NO:3). In some embodiments, the IL3Ra lacks the signal peptide, e.g. comprises amino acids 19-378 of SEQ ID NO:3. In some embodiments, the peptide comprises a sequence that is at least 80%, 85%, 90%, 95%, 97%, or 99% identical to amino acids 1-378 or 19-378 of SEQ ID NO:1.

As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, 15 at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Various other sequence alignment programs are available and can be used to determine sequence identity such as, for example, Clustal.

### Nucleic Acids and Delivery Vectors

In some embodiments, the methods can include administering a nucleic acid (e.g., an mRNA) encoding an IL3R agonist, e.g., an IL3 peptide or daniplestim, or an IL3Ra peptide (e.g., as described herein). Nucleic acids encoding an IL3R agonist can be incorporated into a gene construct to be used as a part of a gene therapy protocol. For example, targeted expression vectors can be used for in vivo delivery and expression of a (optionally codon-optimized) polynucleotide that encodes an IL3R agonist polypeptide or active fragment thereof in particular cell types. For example, IL3 can be expressed in astrocytes, and IL3Ra can be expressed in microglia. Expression constructs of such components can be administered in any effective carrier, e.g., any formulation or composition capable of effectively delivering the component gene to cells in vivo. Approaches include insertion of the gene in viral vectors, preferably adeno-associated virus. Viral vectors typically transduce cells directly.

Viral vectors capable of highly efficient transduction of CNS neurons may be employed, including any serotypes of rAAV (e.g., AAV1-AAV12) vectors, recombinant or chimeric AAV vectors, as well as lentivirus or other suitable viral vectors. In some embodiments, a codon-optimized polynucleotide encoding an IL3R agonist as described herein is operably linked to promoter suitable for expression in the CNS. For example, an astrocyte-specific promoter, such as GFAP or Aldh1l1 promoter can be used to drive expression of IL3 in astrocytes. Alternatively, a microglial promoter, such as CD11b or Iba1 promoter, can be used to drive IL3Ra expression in microglia. Other exemplary promoters include, but are not limited to, a cytomegalovirus (CMV) early enhancer/promoter; a hybrid CMV enhance/chicken β-actin (CBA) promoter; a promoter comprising the CMV early enhancer element, the first exon and first intron of the chicken β-actin gene, and the splice acceptor of the rabbit β-globin gene (commonly call the “CAG promoter”); or a 1.6-kb hybrid promoter composed of a CMV immediate-early enhancer and CBA intron 1/exon 1 (commonly called the CAGGS promoter; Niwa et al. Gene, 108:193-199 (1991)). The CAGGS promoter (Niwa et al., 1991) has been shown to provide ubiquitous and long-term expression in the brain (Klein et al., Exp. Neurol. 176:66-74 (2002)). One approach for in vivo introduction of nucleic acid into a cell is by use of a viral vector containing nucleic acid, e.g., a codon-optimized cDNA encoding an IL3R agonist. Among other things, infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

A viral vector system particularly useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro and Immunol.158:97-129 (1992)). AAV vectors efficiently transduce various cell types and can produce long-term expression of transgenes in vivo. Although AAV vector genomes can persist within cells as episomes, vector integration has been observed (see for example Deyle and Russell, Curr Opin Mol Ther. 2009 Aug; 11(4): 442-447; Asokan et al., Mol Ther. 2012 April; 20(4): 699-708; Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989)). AAV vectors, such as AAV2, have been extensively used for gene augmentation or replacement and have shown therapeutic efficacy in a range of animal models as well as in the clinic; see, e.g., Mingozzi and High, Nature Reviews Genetics 12, 341-355 (2011); Deyle and Russell, Curr Opin Mol Ther. 2009 Aug; 11(4): 442-447; Asokan et al., Mol Ther. 2012 April; 20(4): 699-708. AAV vectors containing as little as 300 base pairs of AAV can be packaged and can produce recombinant protein expression. Protocols for producing recombinant retroviruses and for infecting cells in vitro or in vivo with such viruses are known in the art, e.g, can be found in Ausubel, et al., eds., Current Protocols in Molecular Biology, Greene Publishing Associates, (1989), Sections 9.10-9.14, and other standard laboratory manuals. The use of AAV vectors to deliver constructs for expression in the brain has been described, e.g., in Iwata et al., Sci Rep. 2013;3:1472; Hester et al., Curr Gene Ther. 2009 Oct;9(5):428-33; Doll et al., Gene Therapy 1996, 3(5):437-447; and Foley et al., J Control Release. 2014 Dec 28;196:71-8.

Thus, in some embodiments, the IL3R agonist-encoding nucleic acid is present in a vector for gene therapy, such as an AAV vector. In some instances, the AAV vector is selected from the group consisting of AAV-F, AAV1, AAV2, AAV3, AAV4, AAV5, AAV6, AAV7, AAV8, AAV9, AAVrh10, AAV11, and AAV12. See, e.g., Castle et al., Hum Gene Ther. 2020 Apr;31(7-8):415-422 (intraparenchymal adeno-associated virus serotype 2 (AAV2)); O'Carroll et al., Front Mol Neurosci. 2021 Jan 11;13:618020 (AAV targeting of glial cell types); Hanlon et al., Mol Ther Methods Clin Dev. 2019 Oct 23;15:320-332 (AAV-F, a variant of AAV9); Griciuc A, Federico AN, Natasan J, et al. Gene therapy for Alzheimer's disease targeting CD33 reduces amyloid beta accumulation and neuroinflammation. Hum Mol Genet. 2020;29(17):2920-2935 (AAV9).

A vector as described herein can be a pseudotyped vector. Pseudotyping provides a mechanism for modulating a vector's target cell population. For instance, pseudotyped AAV vectors can be utilized in various methods described herein. Pseudotyped vectors are those that contain the genome of one vector, e.g., the genome of one AAV serotype, in the capsid of a second vector, e.g., a second AAV serotype. Methods of pseudotyping are well known in the art. For instance, a vector may be pseudotyped with envelope glycoproteins derived from Rhabdovirus vesicular stomatitis virus (VSV) serotypes (Indiana and Chandipura strains), rabies virus (e.g., various Evelyn-Rokitnicki-Abelseth ERA strains and challenge virus standard (CVS)), Lyssavirus Mokola virus, a rabies-related virus, vesicular stomatitis virus (VSV), Mokola virus (MV), lymphocytic choriomeningitis virus (LCMV), rabies virus glycoprotein (RV-G), glycoprotein B type (FuG-B), a variant of FuG-B (FuG-B2) or Moloney murine leukemia virus (MuLV). A virus may be pseudotyped for transduction of one or more neurons or groups of cells.

Without limitation, illustrative examples of pseudotyped vectors include recombinant AAV2/1, AAV2/2, AAV2/5, AAV2/6, AAV2/7, AAV2/8, AAV9, AAVrh10, AAV11, and AAV12 serotype vectors. It is known in the art that such vectors may be engineered to include a transgene encoding a human protein or other protein. In particular instances, the present disclosures can include a pseudotyped AAV9 or AAVrh10 viral vector including a nucleic acid as disclosed herein. See Viral Vectors for Gene Therapy: Methods and Protocols, ed. Machida, Humana Press, 2003.

In some instances, a particular AAV serotype vector may be selected based upon the intended use, e.g., based upon the intended route of administration.

Various methods for application of AAV vector constructs in gene therapy are known in the art, including methods of modification, purification, and preparation for administration to human subjects (see, e.g., Viral Vectors for Gene Therapy: Methods and Protocols, ed. Machida, Humana Press, 2003). In addition, AAV based gene therapy targeted to cells of the CNS has been described (see, e.g., U.S. Pat. Nos. 6,180,613 and 6,503,888). High titer AAV preparations can be produced using techniques known in the art, e.g., as described in U.S. Pat. No. 5,658,776.

A vector construct refers to a polynucleotide molecule including all or a portion of a viral genome and a transgene. In some instances, gene transfer can be mediated by a DNA viral vector, such as an adenovirus (Ad) or adeno-associated virus (AAV). Other vectors useful in methods of gene therapy are known in the art. For example, a construct as disclosed herein can include an alphavirus, herpesvirus, retrovirus, lentivirus, or vaccinia virus.

Adenoviruses are a relatively well characterized group of viruses, including over 50 serotypes (see, e.g., WO 95/27071, which is herein incorporated by reference). Adenoviruses are tractable through the application of techniques of molecular biology and may not require integration into the host cell genome. Recombinant Ad-derived vectors, including vectors that reduce the potential for recombination and generation of wild-type virus, have been constructed (see, e.g., international patent publications WO 95/00655 and WO 95/11984, which are herein incorporated by reference). Wild-type AAV has high infectivity and is capable of integrating into a host genome with a high degree of specificity (see, e.g. Hermonat and Muzyczka 1984 Proc. Natl. Acad. Sci., USA 81:6466-6470 and Lebkowski et al. 1988 Mol. Cell. Biol. 8:3988-3996).

Non-native regulatory sequences, gene control sequences, promoters, non-coding sequences, introns, or coding sequences can be included in a nucleic acid as disclosed herein. The inclusion of nucleic acid tags or signaling sequences, or nucleic acids encoding protein tags or protein signaling sequences, is further contemplated herein. Typically, the coding region is operably linked with one or more regulatory nucleic acid components.

A promoter included in a nucleic acid as disclosed herein can be a tissue- or cell type-specific promoter, a promoter specific to multiple tissues or cell types, an organ-specific promoter, a promoter specific to multiple organs, a systemic or ubiquitous promoter, or a nearly systemic or ubiquitous promoter. Promoters having stochastic expression, inducible expression, conditional expression, or otherwise discontinuous, inconstant, or unpredictable expression are also included within the scope of the present disclosure. A promoter can include any of the above characteristics or other promoter characteristics known in the art.

In clinical settings, the gene delivery systems for the therapeutic gene can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection, e.g., optionally into the cisterna magna, cerebral ventricles, lumbar intrathecal space, direct injection into hippocampus (e.g., Chen et al., PNAS USA 91: 3054-3057 (1994)). In preferred embodiments, delivery methods of IL3 agonist-expressing virus include intravenous, intrathecal, intracerebroventricular, intracisternal, and stereotactic intraparenchymal administration.

The methods can be further optimized via preclinical testing to achieve the best rescue of neurodegeneration, dementia, synaptic dysfunction and molecular alteration in animal models.

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

### Delivery Formulations and Pharmaceutical Compositions

In some embodiments, polynucleotides as disclosed herein for delivery to a target tissue in vivo are encapsulated or associated with in a nanoparticle. Methods for nanoparticle packaging are well known in the art, and are described, for example, in Bose S, et al (Role of Nucleolin in Human Parainfluenza Virus Type 3 Infection of Human Lung Epithelial Cells. J. Virol. 78:8146. 2004); Dong Y et al. Poly(d,l-lactide-co-glycolide)/montmorillonite nanoparticles for oral delivery of anticancer drugs. Biomaterials 26:6068. 2005); Lobenberg R. et al (Improved body distribution of 14C-labelled AZT bound to nanoparticles in rats determined by radioluminography. J Drug Target 5:171.1998); Sakuma S R et al (Mucoadhesion of polystyrene nanoparticles having surface hydrophilic polymeric chains in the gastrointestinal tract. Int J Pharm 177:161. 1999); Virovic L et al. Novel delivery methods for treatment of viral hepatitis: an update. Expert Opin Drug Deliv 2:707.2005); and Zimmermann E et al, Electrolyte- and pH-stabilities of aqueous solid lipid nanoparticle (SLN) dispersions in artificial gastrointestinal media. Eur J Pharm Biopharm 52:203. 2001).

The present methods and compositions can include microvesicles or a preparation thereof, that contains one or more IL3R agonist therapeutic molecules, e.g., peptides, polypeptides, polynucleotides or RNA as described herein. “Microvesicles”, as the term is used herein, refers to membrane-derived microvesicles, which includes a range of extracellular vesicles, including exosomes, microparticles and shed microvesicles secreted by many cell types under both normal physiological and pathological conditions. See, e.g., EP2010663B1. The methods and compositions described herein can be applied to microvesicles of all sizes; in one embodiment, 30 to 200 nm, in one embodiment, 30 to 800 nm, in one embodiment, up to 2 um. The methods and compositions described herein can also be more broadly applied to all extracellular vesicles, a term which encompasses exosomes, shed microvesicles, oncosomes, ectosomes, and retroviral-like particles. Such a microvesicle or preparation is produced by the herein described methods. As the term is used herein, a microvesicle preparation refers to a population of microvesicles obtained/prepared from the same cellular source. Such a preparation is generated, for example, in vitro, by culturing cells expressing the nucleic acid molecule of the instant invention and isolating microvesicles produced by the cells. Methods of isolating such microvesicles are known in the art (Thery et al., Isolation and characterization of exosomes from cell culture supernatants and biological fluids, in Current Protocols Cell Biology, Chapter 3, 322, (John Wiley, 2006); Palmisano et al., (Mol Cell Proteomics. 2012 August; 11(8):230-43) and Waldenström et al., ((2012) PLoS ONE 7(4): e34653.doi: 10.1371/journal.pone.0034653)), some examples of which are described herein. Such techniques for isolating microvesicles from cells in culture include, without limitation, sucrose gradient purification/separation and differential centrifugation, and can be adapted for use in a method or composition described herein. See, e.g., EP2010663B1.

In some embodiments, one or more IL3R agonists is delivered to a target tissue in vivo in a vesicle, e.g. a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid). In some embodiments, lipid-based nanoparticles (LNP) are used; see, e.g., Robinson et al., Mol Ther. 2018 Aug 1;26(8):2034-2046; U.S. Pat. No. 9,956,271B2.

In some embodiments, the microvesicles are isolated by gentle centrifugation (e.g., at about 300 g) of the culture medium of the donor cells for a period of time adequate to separate cells from the medium (e.g., about 15 minutes). This leaves the microvesicles in the supernatant, to thereby yield the microvesicle preparation. In one embodiment, the culture medium or the supernatant from the gentle centrifugation, is more strongly centrifuged (e.g., at about 16,000 g) for a period of time adequate to precipitate cellular debris (e.g., about 30 minutes). This leaves the microvesicles in the supernatant, to thereby yield the microvesicle preparation. In one embodiment, the culture medium, the gentle centrifuged preparation, or the strongly centrifuged preparation is subjected to filtration (e.g., through a 0.22 um filter or a 0.8 um filter, whereby the microvesicles pass through the filter. In one embodiment, the filtrate is subjected to a final ultracentrifugation (e.g. at about 110,000 g) for a period of time that will adequately precipitate the microvesicles (e.g. for about 80 minutes). The resulting pellet contains the microvesicles and can be resuspended in a volume of buffer that yields a useful concentration for further use, to thereby yield the microvesicle preparation. In one embodiment, the microvesicle preparation is produced by sucrose density gradient purification. In one embodiment, the microvesicles are further treated with DNAse (e.g., DNAse I) and/or RNAse and/or proteinase to eliminate any contaminating DNA, RNA, or protein, respectively, from the exterior. In one embodiment, the microvesicle preparation contains one or more RNAse inhibitors.

The molecules contained within the microvesicle preparation will comprise the therapeutic molecule. Typically the microvesicles in a preparation will be a heterogeneous population, and each microvesicle will contain a complement of molecule that may or may not differ from that of other microvesicles in the preparation. The content of the therapeutic molecules in a microvesicle preparation can be expressed either quantitatively or qualitatively. One such method is to express the content as the percentage of total molecules within the microvesicle preparation. By way of example, if the therapeutic molecule is an mRNA, the content can be expressed as the percentage of total RNA content, or alternatively as the percentage of total mRNA content, of the microvesicle preparation. Similarly, if the therapeutic molecule is a protein, the content can be expressed as the percentage of total protein within the microvesicles. In one embodiment, therapeutic microvesicles, or a preparation thereof, produced by the method described herein contain a detectable, statistically significantly increased amount of the therapeutic molecule as compared to microvesicles obtained from control cells (cells obtained from the same source which have not undergone scientific manipulation to increase expression of the therapeutic molecule). In one embodiment, the therapeutic molecule is present in an amount that is at least about 10%, 20%, 30% 40%, 50%, 60%, 70% 80% or 90%, more than in microvesicles obtained from control cells. Higher levels of enrichment can also be achieved. In one embodiment, the therapeutic molecule is present in the microvesicle or preparation thereof, at least 2 fold more than control cell microvesicles. Higher fold enrichment can also be obtained (e.g., 3, 4, 5, 6, 7, 8, 9 or 10 fold).

In one embodiment, a relatively high percentage of the microvesicle content is the therapeutic molecule (e.g., achieved through overexpression or specific targeting of the molecule to microvesicles). In one embodiment, the microvesicle content of the therapeutic molecule is at least about 10%, 20%, 30% 40%, 50%, 60%, 70% 80% or 90%, of the total (like) molecule content (e.g., the therapeutic molecule is an mRNA and is about 10% of the total mRNA content of the microvesicle). Higher levels of enrichment can also be achieved. In one embodiment, the therapeutic molecule is present in the microvesicle or preparation thereof, at least 2 fold more than all other such (like) molecules. Higher fold enrichment may also be obtained (e.g., 3, 4, 5, 6, 7, 8, 9 or 10 fold).

The methods described herein can include pharmaceutical compositions comprising or consisting of 1L3$ agonists as an active ingredient, and methods for use thereof for treating subjects who have AD.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Materials and Methods

The following materials and methods were used in the examples set forth below.

Human Samples. Frozen tissue specimens and paraffin sections from the frontal cortex of AD patients and age-matched non-demented control subjects were obtained from the Massachusetts Alzheimers Disease Research Center Brain Bank. Subjects or next of kin consented to the brain donation and the Massachusetts General Hospital Institutional Review Board approved the study. All AD patients met the National Institute of Neurological and Communicative Disorders and Stroke-Alzheimer's Disease and Related Disorders Associations criteria for probable AD and the National Institute on Aging-Reagan Institute criteria for high likelihood of AD. Secondary use of de-identified human samples was approved by the institutional review board of the Massachusetts General Hospital (protocol no. 2019P003736 and 2019P003732).

Animals. Wild-type C57BL/6J, B6;FVB-Tg(Aldh1l1-EGFP/Rpl10a)JD130Htz/J, C57BL/6J-Trem2em2Adiuj/J, and C57BL/6-Tg(UBC-GFP)30Scha/J mice were purchased from The Jackson Laboratory. 5×FAD mice²⁸ were purchased from the Jackson Laboratory (MMRRC) and backcrossed onto the C57BL/6J background more than 10 generations before being crossed with other strains. Il3^{-/-} mice on the C57BL/6J background were bred in-house^{21,29} and crossed with 5×FAD mice. For RNAseq studies, Trem2^{-/-} mice³⁰ on the C57BL/6J background were generated at Washington University School of Medicine, bred in-house, and crossed with 5×FAD mice. Age- and sex-matched animals were used. If sex of the animals is not specifically indicated, groups were sex balanced. Where appropriate, animals were randomly assigned to interventions. All mice were group housed under standard conditions with free access to food and water. All animal protocols were approved by the Animal Review Committee at the Massachusetts General Hospital (protocol no. 2011N000035 and 2015N000044) and were in compliance with relevant ethical regulations.

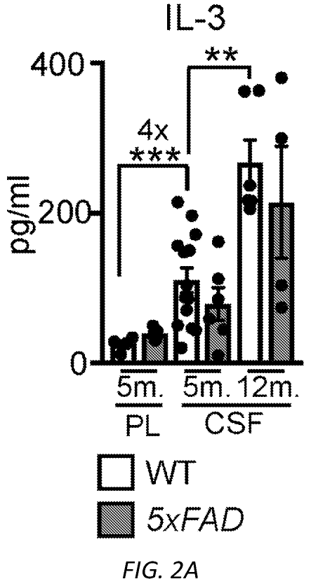

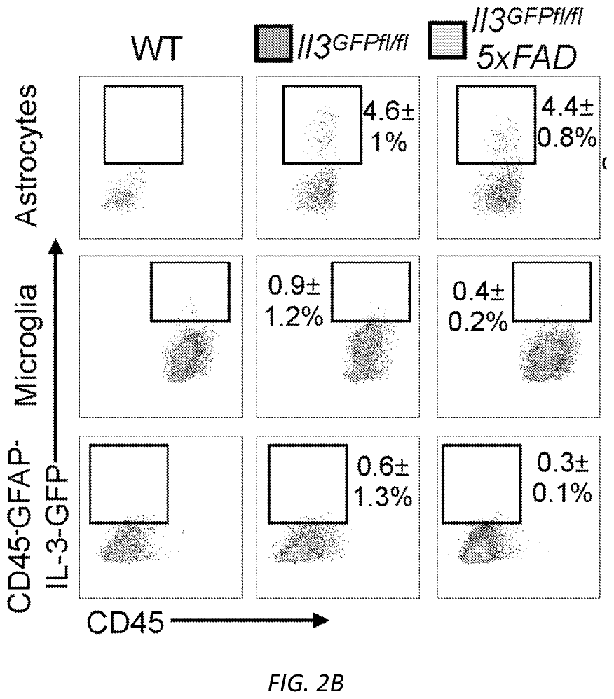

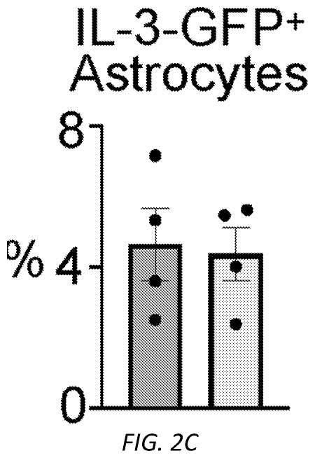

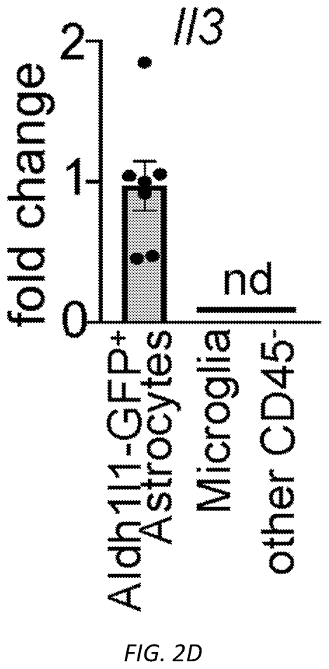

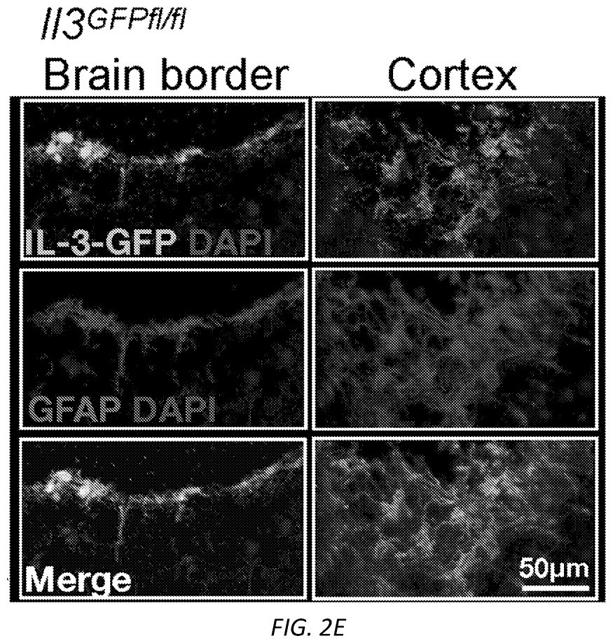

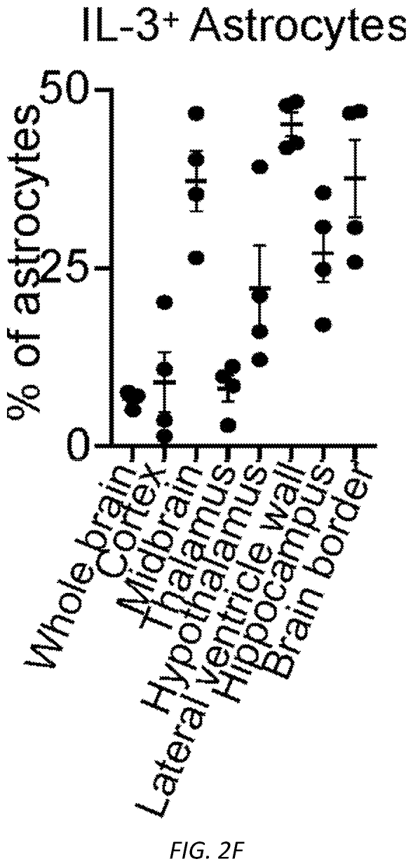

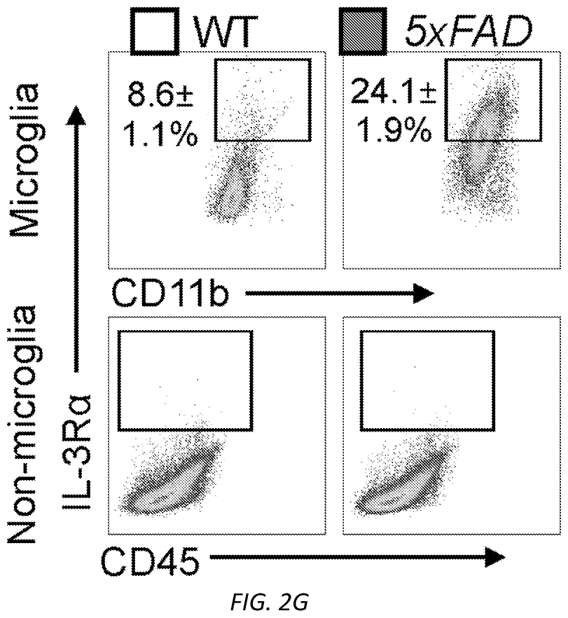

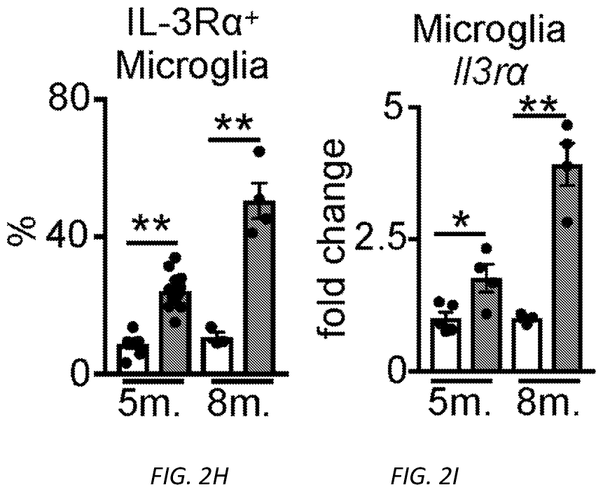

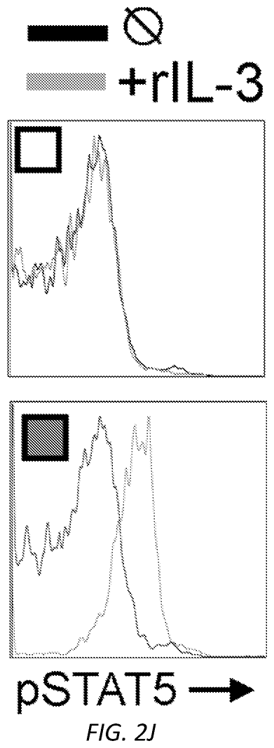

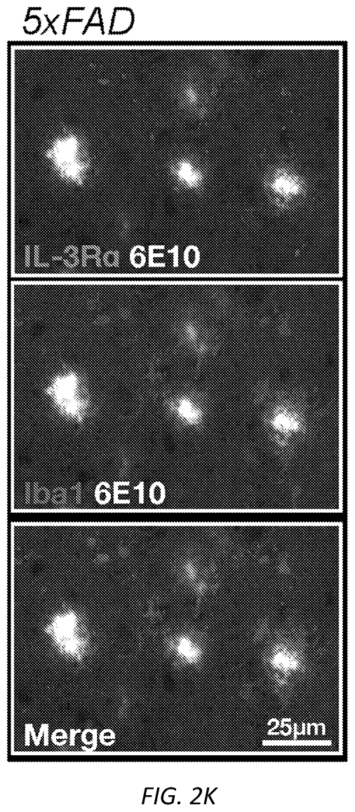

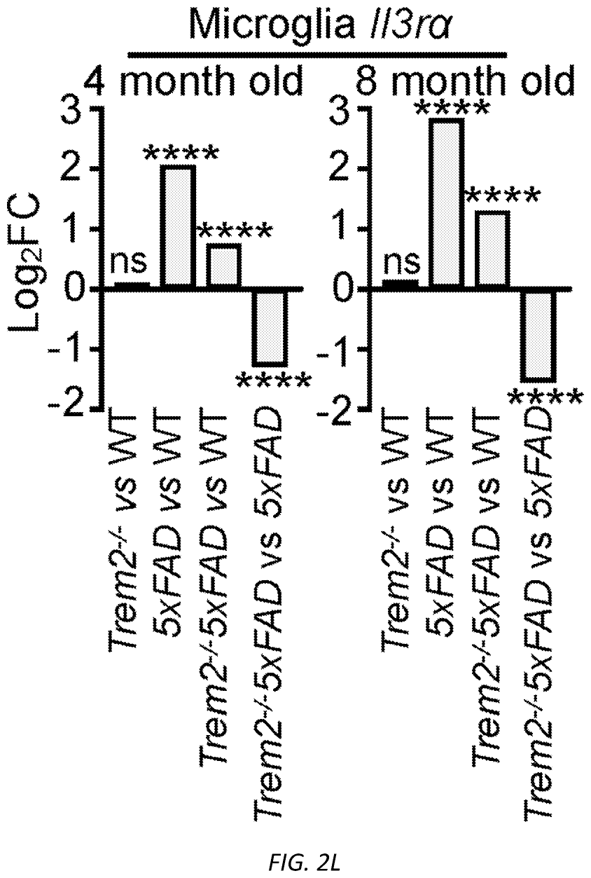

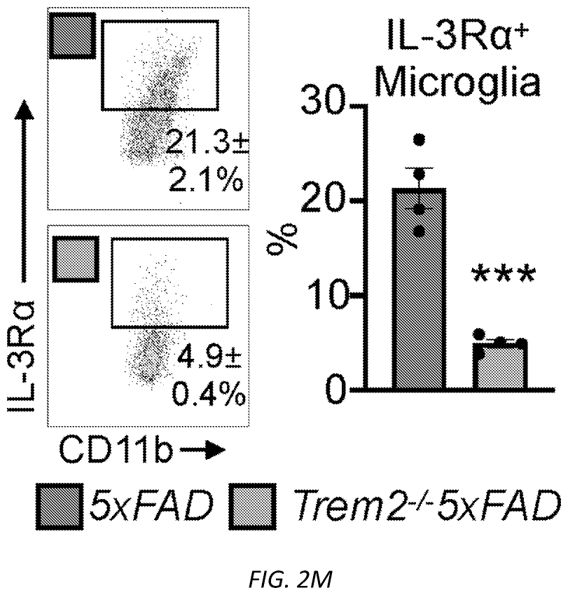

CRISPR-Cas9 generation of Il3^{GFPfl/fl} and Il3ra^{fl/fl} mice. Two SpCas9 guide RNAs (gRNAs; Table 1) were initially designed to target genomic regions within the first intron and 3′ of the stop codon of either Il3 and Il3Ra genes, using on-target and off-target prediction software^{31,32}. Single stranded DNA (ssDNA) donor oligos encoding the floxed cDNA were designed for Il3 and Il3ra, both of which encoded a P2A-eGfp tag and ˜500 base pair homology arms on either end (synthesized by Genewiz). Prior to performing experiments with the ssDNA donors, the on-target activities of the gRNAs were evaluated by microinjection of ribonucleoprotein (RNP) complexes comprised of TrueCut Cas9 v2 (ThermoFisher) and synthetic gRNAs (Synthego) into mouse zygotes. All microinjections were performed at the Genome Modification Facility (Harvard University). Injected zygotes developed to the blastocyst stage prior to genomic DNA extraction. To evaluate genome editing efficiencies, the target regions were amplified by PCR using the primers listed in Table 2. Amplicons were sent for Sanger sequencing and the approximate level of on-target activity was determined using ICE³³. The most effective gRNA of each pair examined (within the first intron and 3′ of the stop codon of either Il3 and Il3Ra genes) were then used for microinjections in the presence of the ssDNA donors. Injected embryos were implanted into pseudopregnant recipients, and 17 and 24 pups for the Il3 and Il3Ra targeted mice, respectively, were genotyped at 3 weeks of age. To genotype mice, genomic DNA was extracted from tail snips in 200 μL of tail lysis buffer (100 mM Tris-HCl, 200 mM NaCl, 5 mM EDTA, 0.05% SDS, 12.5 mM DTT, 1.4 ug/ul Proteinase K (New England Biolabs)) via ˜16 hour incubation at 55° C. Lysates were cleaned up using 0.7× paramagnetic beads prepared as previously described^{34,35}. Insertions of the donor DNA sequences into the endogenous Il3 and Il3Ra loci were confirmed by Sanger sequencing across the full donor sequence (using the primers in Table 3). Founder mice in which the full-length insert was detected were then selected for further breeding to remove mosaicism and generate Il3^{GFPfl/fl} and Il3ra^{fl/fl} N1 mice. Sanger sequencing revealed missense mutations in the inserted sequence that were not present in the ssDNA donor sequence (Table 4). Missense mutations resulted in the quenching of the GFP signaling in Il3ra targeted mice but did not influence IL-3Rα functionality or signaling. GFP functionality remained intact in Il3 targeted mice (FIG. 2 b and f) Subsequent Il3^{GFPfl/fl} and Il3ra^{fl/fl} mice were genotyped by PCR via the genotyping strategy and primers in Table 5. Il3^{GFPfl/fl} mice were crossed to Aldh1l1Cre^{ERT2} and 5×FAD mice generating Il3^{GFPfl/fl}Aldh1l1Cre^{ERT2} 5×FAD mice, while Il3ra^{fl/fl} mice were crossed to Cx3cr1Cre^{ERT2} and 5×FAD mice generating Il3ra^{fl/fl}CX3Cr1Cre^{ERT2} 5×FAD mice.

In vivo interventions. Parabiosis. The procedure was conducted as previously described³⁶. In brief, age-, sex, and weight-matched animals were used and housed together for a least 14 days prior to surgery. The corresponding lateral aspects of each mouse were shaved, incisions were made from the forelimb joint to the hindlimb joint and the subcutaneous fascia was bluntly dissected to create 0.5 cm of free skin. Fore- and hindlimb joints were joined and the dorsal and ventricle skins were approximated by continuous suture using mononylon 5.0 (Ethicon).

LPS injection. Mice were injected daily intraperitoneally with 20 μg lipopolysaccharide (LPS, Sigma) for 4 days.

BrdU injection. Mice were injected intraperitoneally with 1.5 mg of BrdU (Sigma) twice a day for 5 days.

Recombinant IL-3 brain infusion. Cannula and osmotic minipump (Alzet) implantation were performed as previously described³⁷. Briefly, mice were anesthetized, the head was shaved and secured in a stereotactic frame (Stoelting). An incision was made above the skull extending behind the shoulder blades. A small hole was drilled in the skull at AP −1; ML −0.27 from bregma and depth 2 mm from dura to target the lateral ventricle. The cannula was inserted and glued to the skull. The cannula was connected to an osmotic minipump filled with recombinant IL-3 (Biolegend) conjugated to an anti-IL-3 antibody (Biolegend) as previously described²⁹. Minipumps delivered rIL-3 into the ventricle at a rate of 1 μg/day. Minipumps were implanted subcutaneously caudal the shoulder blades. At the end of the procedure the incision was sutured using mononylon 5.0 (Ethicon).

Steriotactic injection. Mice were anesthetized, the head was shaved and secured in a stereotactic frame (Stoelting). An incision was made above the skull and a hole was drilled at AD −0.1; ML −0.1 from Bregma and depth 0.1 mm from dura to target the cortex. Using a 0.5 μl Hamilton syringe 3 μg of interleukin-3 (Biolegend) conjugated to an anti-interleukin-3 antibody (Biolegend) was delivered in a volume of 0.5 μl. Regions of the cortex a minimum 600 μm away from the injection site were analyzed.

Peripheral rIL-3 injection. Mice were injected intraperitoneally with 10 μg of recombinant interleukin-3 (Biolegend) conjugated to an anti-interleukin-3 antibody (Biolegend) twice a week for 10 weeks.

Cerebrospinal fluid collection. Mice were anaesthetized and the skin of the neck was shaved and disinfected with 70% ethanol. Mice were placed in a stereotactic frame (Stoelting) to secure their heads. A skin incision was made at the back of the neck and muscle layers were retracted to expose the cisterna magna. Cerebrospinal fluid was collected by piercing the pia mater with a microcapillary tube (VWR) and allowing CSF to collect in the capillary.

FITC-dextran injection. 4 hours prior to sacrifice mice were injected i.v. with FITC-Dextran (mol. wt. 4000, Sigma Aldrich). At sacrifice mice were perfused at a rate of 5 ml/min with 20 ml PBS. Brain tissue was homogenized and FITC signal was measured by spectrophotometry in tissue supernatant.

PE-GR1 injection. 4 hours prior to sacrifice mice were injected i.v. with an anti-GR1 antibody conjugated to PE (Biolegend). At sacrifice mice were perfused with 10 ml PBS and the leukocyte fraction was isolated from brain tissue prior to flow cytometry analysis.

Tamoxifen injection. 20 mg/ml tamoxifen (Sigma Aldrich) was prepared in corn oil and allowed to dissolve at 37° C. overnight while shaking. Mice were injected i.p. with 2 mg tamoxifen on 4 consecutive days at 2 months of age then monthly thereafter until sacrifice at 5 months of age.

Cells. Mouse cell collection. Peripheral blood was collected by retro-orbital bleeding and red blood cells were lysed in RBC lysis buffer (Biolegend). Bone marrow cells were collected by flushing bones with PBS, after which a single-cell suspension was created by passing cells through a 26-gauge needle and red blood cells were lysed with RBC lysis buffer. Brain was excised after PBS (Thermo Fisher Scientific) perfusion, minced and digested with 450 U ml⁻¹ collagenase I, 125 U ml⁻¹ collagenase XI, 60 U ml-¹ DNase I and 60 U ml⁻¹ hyaluronidase (Sigma) in PBS for 40 min at 37° C. Samples were passed through a 70-μm cell strainer and mixed with 30% percol layered on-top of 70% percol. The percol gradient was centrifuged at 500 g for 30 mins with brake off. The cell fraction was collected and washed with PBS before downstream applications. Total viable cell numbers were counted using trypan blue (Cellgro, Mediatech) or counting beads (Thermo Fisher Scientific).

Mouse flow cytometry. Single-cell suspensions were stained in PBS supplemented with 2% FBS and 0.5% BSA. The following monoclonal antibodies were used for flow cytometry analyses at a dilution of 1/700 unless otherwise indicated: anti-CD45 (BioLegend, clone30-F11, 103147), anti-CD3 (BioLegend, clone 17A2, 100206), anti-CD90.2 (BioLegend, clone 53-2.1, 105308), anti-CD19 (BioLegend, clone 6D5, 115508), anti-B220 (BD Biosciences, clone RA3-6B2, 553089), anti-NK1.1 (BioLegend, clone PK136, 108708), anti-Ly-6G (BioLegend, clone 1A8, 127614), anti-Ly-6C (BioLegend, AL-21, 128006), anti-MHCII (BioLegend, clone M5/114.152, 107602), anti-CD11b (BioLegend, clone M1/70, 101226), anti-CD115 (BioLegend, clone AFS98, 135517), anti-Ter119 (BioLegend, clone TER-119, 116208), anti-CD34 (eBioscience, clone RAM34, 11-0341-85), anti-CD49b (BioLegend, clone DX5, 1089008), anti-CD11c (BioLegend, clone N418, 117310), anti-IL-7Rα (BioLegend, clone SB/199, 121112), anti-CD16/32 (BioLegend, clone 93, 101324), anti-CD150 (BioLegend, clone TC15-12F12.2, 115922), anti-cKit (BioLegend, clone 2B8, 105814), anti-CD135 (BioLegend, clone A2F10, 135310), anti-CD48 (BioLegend, clone HM48-1, 103426), anti-Sca1 (BioLegend, clone D7, 108126), anti-IL-3 (1/100, BD Bioscience, clone MP2-8F8, 55483), anti-IL-3Rα (eBioscience, clone 6H6, 14-1239-82), anti-GFAP (eBioscience, GSA, 53-982-80), anti CCL2 (eBioscience, clone 2H5, 11-7096-81), anti-β-amyloid (BioLegend, clone 6E10, 803013), anti-TREM2 (R&D Systems, clone 237920, FAB17291P), anti-CD11c (BioLegend, clone N418, 117333), anti-BrdU (eBioscience, clone BU20A, 17-5071-42). All antibodies were used in a 1:700 dilution except IL-3 and IL-3Ra which was used at a 1:100 dilution. BrdU staining and intracellular staining were performed using a commercial kits according to manufacturers instructions (BD Bioscience). Viable cells were identified as unstained with Zombie Aqua (BioLegend) or 7AAD (BioLegend). Data were acquired on a LSRII (BD Biosciences) and analyzed with FlowJo (Tree Star).

Mouse flow cytometry gating. Live, singlet cells were identified as (1) Ly-6C^{high} monocytes (CD45⁺CD11b⁺CD115⁺Ly-6C^{high}), (2) neutrophils (CD45⁺CD11b⁺Ly-6G⁺), (3)Bcells(CD45⁺B220⁺CD19⁺CD11b⁻), (4) T cells (CD45⁺CD3⁺CD90⁺CD11b⁻), (5) LSK cells (CD45⁺Lin⁻Kit⁺Sca1⁺), (6) multipotent progenitor (MPP)4 (CD45⁺Lin⁻Kit⁺Sca1⁺CD135⁺CD150⁻), (7) MPP3 (CD45⁺Lin⁻Kit⁺Sca1⁺CD135⁺CD150⁻CD48⁺), (8) short-term hematopoietic stem cells (CD45⁺Lin⁻Kit⁺Sca1⁺CD135⁺CD150⁻CD48⁻), (9) long-term hematopoietic stem cells (CD45⁺Lin⁻Kit⁺Sca1⁺CD135⁺CD150⁺CD48⁻), (10) common myeloid progenitor (CD45⁺Lin⁻Kit⁺Sca1⁻CD34⁺CD16/32^{mid}), (11) granulocyte-macrophage progenitor (CD45⁺Lin⁻Kit⁺Sca1⁺CD34⁺CD16/32^{high}CD115⁻), (12) monocyte-dendritic cell progenitor (CD45⁺Lin⁻Kit⁺Sca1⁺CD34⁺CD16/32^{high}CD115⁺), (13) Microglia (CD45^{mid}CD11b⁺), (14) Astrocytes (CD45⁻CD11b⁻GFAP⁻ or CD45⁻CD11b⁻Aldh1l1-GFP⁺), (15) Other brain cells (CD45⁻CD11b⁻GFAP⁻ or CD45⁻CD11b⁻Aldh1l1-GFP⁻). Lin=B220, CD19, CD49b, Ter119, CD90.2, CD11b, CD11c, Ly6G, IL1Rα.

Cell sorting. Brain cell suspensions were stained to identify the indicated cell populations and cells were sorted on a FACS Aria II cell sorter (BD Biosciences) directly into collection medium.

Ex-vivo cell cultures. Microglia. Sorted microglia were cultured in complete medium (RPMI-1640 supplemented with 10% FBS, 2 mM L-glutamine, 10 Uml⁻penicillin and streptomycin, 10 mM HEPES, 50 μM 2-mercaptoethanol, 1 mM sodium pyruvate and 1× nonessential amino acids) and kept in humidified 5% CO₂ incubator at 37° C. Microglia were exposed to 20 ng/ml recombinant IL-3 (Biolegend) and/or 2 μg/ml Beta-Amyloid (1-42) HiLyte™ conjugated to pHrodo iFL red (Invitrogen) for 3 hours. T-cells. Naive T cells were isolated from the spleen and lymph nodes using a Naive T cell isolation kit (Miltenyi Biotec) and cultured on anti-CD3 (2 μg/mL) coated plates in the presence of soluble anti-CD28 (2 μg/mL) and rmIL-2 (10 μg/mL) for 3 days and re-stimulated with PMA (100 ng/mL) and ionomycin (500 ng/mL) in the presence of GolgiPlug and GolgiStop (1:1000) for 3.5 hours prior to cell surface staining and analysis.

RNA-seq: WT, Trem2^{-/-}, 5×FAD, and Trem2^{-/-}5×FAD mice. Microglia isolation and FACS sorting. Microglia were isolated from 4 and 8-month-old WT, Trem2^{-/-}, 5×FAD and 5×FAD;Trem2^{-/-} mice as previously described²⁴. Briefly, mice were deeply anesthetized with CO₂ and transcardially perfused with PBS/1 mM EDTA. Brains were placed into a GentleMacs C-tube (Miltenyi Biotech) with pre-warmed RPMI 1640 medium (Gibco) containing Dispase (2 U/ml), and Collagenase Type 3 (200 U/ml, Worthington Biochemical Corporation). Using the GentleMACS Dissociator (Miltenyi Biotech), brains were subjected to three rounds of dissociation, each followed by a period of incubation at 37° C. After the second round of dissociation, DNase I grade II (Roche) was added to a final concentration of 40 U/ml and incubated at 37° C. After the third round of dissociation, the enzymes were inactivated by adding PBS containing 2 mM EDTA and 5% fetal bovine serum. The brain tissue was triturated, passed through a 100-μm filter (Thermo Fisher Scientific) and centrifuged. Cell pellets were resuspended in 10.5 ml RPMI 1640 medium (Gibco), mixed gently with 4.5 ml physiologic Percoll (Sigma), and centrifuged at 850 g for 40 minutes. Subsequently, cells were rinsed with PBS/1 mM EDTA and centrifuged at 500 g for 8 minutes. Contaminating red blood cells were lysed with Red blood cell lysing buffer (Sigma). Cells were rinsed with PBS/1 mM EDTA and centrifuged at 500 g for 8 minutes. Cell pellets were resuspended in blocking buffer (PBS/1 mM EDTA/2% donkey serum) containing Fc block (1 μg/ml, anti-mouse CD16/32, clone 93, Biolegend) and incubated in ice for 10 minutes. Then, cells were labeled with Alexa647-anti-CD11b (5 μg/ml, clone M1/170, Biolegend) and Alexa488-anti-CD45 (5 μg/ml, clone 30-F11, Biolegend) antibodies for 30 minutes on ice. Cells were rinsed and centrifuged at 400 g for 8 minutes. Cells were resuspended in PBS/1.0 mM EDTA and sorted based on CD11b/CD45 expression using FACS ARIA (BD Biosciences). FACS-sorted cells were centrifuged at 600 g for 10 minutes and cell pellets were used for RNA extraction.

RNA purification. RNA purification from microglial samples and mRNA sequencing were performed as previously described²⁴. Briefly, microglial cell pellets were lysed in RLT-Plus buffer (Qiagen) containing 1% β-mercaptoethanol. Cell lysates were transferred to QIAshredder (Qiagen) for homogenization and centrifuged at 18,000 g for 2 minutes. RNA was isolated using the RNeasy Plus Micro Kit (Qiagen). During the RNA extraction protocol, samples were treated with RNase-free DNase I (Qiagen) directly on the RNeasy spin columns at room temperature for 15 minutes and washed with buffer RW1 (Qiagen). Each RNA sample was eluted in RNase-free water (15 μl, Qiagen) and RNA integrity was assessed with the Agilent RNA 6000 Pico Chip on the 2100 Bioanalyzer (Agilent). Purified RNA was quantified using the Qubit RNA High Sensitivity Assay Kit (Invitrogen) on the Qubit Fluorometer 3.0 (Thermo Fisher Scientific). Microglial RNA samples originating from mice of the same genotype, sex and age were pooled as needed to generate samples containing 100 ng of RNA. cDNA libraries were prepared using the TruSeq Stranded mRNA LT Prep Kit (Illumina). The protocol consisted of mRNA purification with poly-T-oligo-attached magnetic beads, mRNA fragmentation, first and second strand cDNA synthesis, 3′end adenylation, adapter ligation, and PCR amplification (11 cycles). Libraries were enriched using the Agencourt AMPure XP beads (Beckman Coulter). cDNA libraries were validated using the Agilent DNA 1000 kit on the 2100 Bioanalyzer (Agilent) and quantified by qPCR before sequencing. Libraries were sequenced on a HiSeq 2500 instrument (Illumina) at the MGH Next Generation Sequencing Core Facility, using single-end 50 bp sequencing.

RNA-seq analysis. RNA sequencing resulted in 48.7 million reads per sample on average as previously described²⁴. The raw reads of the sequencing data were submitted to NCBI-GEO: GSE132508. The splice-aware alignment program STAR was used to map sequencing reads (fastqs) to the mouse (mm10) reference genome. Gene expression counts were calculated using the program HTSeq based on the latest Ensembl annotation for mm10/GRCm38. The R package edgeR was used to make differential gene expression calls from these counts at a two-fold cut-off and false discovery rate (FDR)<0.05 threshold. Gene expression was considered upregulated if log₂FC>1 or downregulated if log₂FC<−1 [FC=fold-change of reads per kilobase per million (RPKMs)] at FDR<0.05. To extract expression data for genes of interest, we used the Python Data Analysis Library (Pandas), a powerful tool for indexing and parsing large data frames.

RNA-seq: 5×FAD and Il3^{-/-}5×FAD mice. Microglia were FACS sorted from brains of 5-month-old animals as described above. Microglia cells were isolated from 12 5×FAD mice (6M/6F) and 12 Il3^{-/-}5×FAD mice (6M/6F). Within each genotype samples from 2M and 2F were pooled generating 3 samples from 5×FAD mice and 3 from Il3^{-/-}5×FAD mice from which RNA was isolatedd the RNA-seq performed. RNA was isolated using E.Z.N.A micro elute total RNA kit according to the manufacturer's instructions (Omega Biotek). cDNA libraries were prepared using the TruSeq Stranded mRNA LT Prep Kit (Illumina). Libraries were sequenced on a HiSeq 2500 instrument (Illumina) at the MGH Next Generation Sequencing Core Facility, using paired-end 50 bp sequencing. Sequencing reads were mapped in a splice-aware fashion to the Ensembl annotation of the mouse GRCm37/mm9 transcriptome. Read counts over transcripts were calculated using HTseq followed by differential expression analysis using EdgeR. Genes were classified as differentially expressed based on the cutoffs of fold change (FC)>1.6, false discovery rate (FDR)<0.1, and p<0.005.

Gene enrichment analysis. Gene enrichment analysis was done using Enrichr (maayanlab.cloud/Enrichr/) with default parameters.

Human iPS triculture microfluidic system. Microfluidic device fabrication. Negative photoresists SU-8 10 and SU-8 100 (MicroChem, Newton, MA, USA) were sequentially patterned using standard lithography on a 4-inch (10.16-cm) silicon wafer to create a mold for cell migration channels of 10 μm height and central/side chambers of 100 μm height. The base and a curing agent were mixed at a 10:1 weight ratio (SYLGARD 184 A/B, Dow corning), poured onto the SU-8 mold, and cured for 1 hour at 25° C. under vacuum and, subsequently, cured for more than 3 hours in an oven at 80 ° C. The cured poly dimethyl-siloxane (PDMS) replica was peeled off the mold and 4 mm holes were punched for cell-containing chambers. PDMS and glass slide assembled, irreversibly, using oxygen plasma at 50 mW, 5 cm, for 30 seconds (PX-250, March Plasma Systems). Immediately after the bonding, 50 μl of diluted BD Matrigel (1:100, BD Biosciences) in DMEM/F12 were injected into each holes and incubated for 2 hours at 25° C. to promote cellular adhesion. The PLL-treated surface was rinsed with autoclaved and 0.2-μm filtered water (AM9920, Life Technologies).

3D cell cultures and differentiation of neural progenitor cells (NPCs). ReN cell VM human neural progenitor cells (NPCs) with or without the K670N/M671L (Swedish) and V717I (London) familial AD mutations were purchased from EMD Millipore. AD mutations result in overproduction of Aβ and neurofibrillary tangle (NFT) p-tau. For 3D cultures, BD Matrigel (BD Biosciences) was mixed with the cells (1×10⁶ cells per mL). The final cell concentration for the mixture was approximately 5×10⁴ cells per mL (1:5 3D thin-culture). We then transferred 10 μl of cell mixtures into the microfluidic device using prechilled pipettes. The microfluidic devices were incubated for 1 hour at 37° C., during gel solidification and then 100 μl differentiation media added^{26,27}. Differentiation media was composed of DMEM/F12 (Life Technologies) media supplemented with 2 mg heparin (StemCell Technologies), 2% (v/v) B27 neural supplement (Life Technologies), 20 mg EGF (Sigma), 20 mg bFGF (Stemgent), and 1% (v/v) penicillin/streptomycin/amphotericin-B solution (Lonza). The 3D-plated cells were differentiated for 4 weeks; media was changed every 3-4 days.

Microglia preparation. To generate induced microglia-like cells (iMGLs), iPSCs (RUID: FA0000030, Cell Line ID: NH50163) obtained from NINDS iPSC cell repository (distributed through RUCDR, https://www.rucdr.org). Briefly, improved and simplified differentiation of iPSCs to CD43⁺ primitive hematopoietic progenitor cells (HPCs) has been achieved by using Stem Cell Technologies STEMdiff™ Hematopoietic Kit (Catalog # 05310)³⁸. On day −1, feeder-free iPSCs that have been expanded in TeSR-E8 media are passaged with ReLeaSR (STEMCELL Technologies) into mTeSR E8 medium with 0.5 μM Thiazovivin onto matrigel coated (1 mg/mL) 6-well plates (Corning Costar). Small aggregates of ˜100 cells each are plated at 10-20 aggregates per cm². Continue to supplement medium B (1 mL) until day 24. On day 25, cells are centrifuged leaving 1 mL conditioned media per 35 mm well. On day 25, cells are re-suspended in microglia media plus 100 ng/mL IL-34, 50 ng/mL TGEβ1, 25 ng/mL M-CSF, 100 ng/mL CD200 and 100 ng/mL CX3CL1 to further mature microglia and ensure homeostasis. On day 27, microglia media with the five cytokine cocktails is added (1 mL per well). Before the experiment, cells were incubated with CellTracker Deep Red Dye (10 μM in DMSO, C34565, Invitrogen) for 30 minutes and washed using medium without serum. After centrifugation (200 g for 5 min), the cells were resuspended in 1 mL of microglia media (1×10⁶ cells/mL). We injected 10 μL of the cell suspension into each side chamber and 100 μL of a culturing medium was added into side chambers. The loaded 3D microdevices were then incubated at 37° C. supplied with 5% CO2.

Recombinant IL-3 treatment. For treatment with human recombinant IL-3 (Abcam), NPC differentiation media containing 15 μg of IL-3 was added to 3-week differentiated 3D culture in central chamber. Recombinant IL-3 was maintained in the media for an additional 2 weeks throughout the migration experiment.

Time-lapse imaging. After microglia loading, cells were recorded using time-lapse imaging using a fully automated Nikon C2 s confocal laser scanning microscope (Nikon Instruments Inc.) with a heated incubator to 37° C. and 5% CO2 (20× magnification; Micro Device Instruments, Avon, MA, USA).

Flow cytometry. Cells of the central chamber were collected and repeatedly pipetted in media to break up Matrigel. Cells were centrifuged and washed with PBS. The single cell suspensions were stained with antibodies in PBS. The following monoclonal antibodies were used at a dilution of 1:700 for flow cytometry analyses: anti-mouse/human CD45 (BioLegend, clone30-F11, 103147), anti-mouse/human CD11b (BioLegend, clone M1/70, 101226). CountBright™ absolute counting beads (Invitrogen) were added to the cell suspension to enumerate cells. Samples were run on Microglia were identified as live CD11b⁺CD45⁺ cells. Data were acquired on a LSRII (BD Biosciences) and analyzed with FlowJo (Tree Star).

MSD ELISA Aβ and chemokines measurement. Levels of Aβ38, Aβ40 and Aβ42 in media were simultaneously measured by a multi-array electrochemiluminescence assay kit (K15200E-2, V-PLEX Aβ Peptide Panel 1 (6E10) kit, Meso Scale Diagnostics (MSD)). To quantify Aβ levels in differentiation media, conditioned media from central chamber was collected at each condition, diluted 1:6 with MSD dilution buffer, and analyzed using the assay kit. Human Chemokine Array (K15047G-1, MSD) kit was used to simultaneously detect relative expression levels of 10 human chemokines. Conditioned media (20 μl from each sample) were collected and analyzed following manufacturer's protocol.

Immunostaining. For immunofluorescent stains, we rinsed the cells and 3D cultures twice with PBS (phosphate-buffered saline). Cells were then fixed through at room temperature (30 min incubation in fresh 4% paraformaldehyde aqueous solution (157-4, ElectronMicroscopy Sciences) followed by rinsing twice with PBS. Cells were permeabilized through incubation in 0.1% Triton X-100 in PBST (phosphate-buffered saline with 0.1% Tween 20) for 15 min at RT. Cell-specific binding was blocked through overnight incubation in 3% human serum albumin in PBST at 4° C. After 24-h incubation with the primary antibody solutions at 4° C., the cells were washed five times. The following antibodies (and dilutions) were used: anti-PHF (1:1,000, A gift from P. Davies, Albert Einstein College of Medicine), anti-GFAP (1:500, Millipore), anti-P2RY12 (1:400, Sigma), anti-IL3Rα (1:200, Biolegend), anti-beta-tubulin III (1:200, Abcam) and anti-IL-3 (1:200, Invitrogen).

Histology. Mouse. Brains were harvested from 5×FAD and Il3^{-/-}5×FAD mice and fixed in 10% formalin overnight. The fixed brains were paraffin-embedded and sectioned in the sagittal plane. The paraffin-embedded sections were deparaffinized and rehydrated prior to immunofluorescent staining. Heat induced antigen retrieval was performed using Retrievagen A (pH6.0) (550524, BD Biosciences), and the sections were permeabilized with 0.3% Triton X-100 in PBS for 10 minutes at room temperature. After the sections were blocked with 4% normal goat serum in PBS, primary antibodies, Iba-1 (1:200, 019-19741, FUJIFILM Wako Chemicals) and Alexa Fluor 488 anti-β-Amyloid, 1-16 (1:250, 803013, 6E10, BioLegend), were incubated at 4° C. overnight. A biotinylated goat anti-rabbit IgG secondary antibody and streptavidin DyLight 594 (1:100, BA-1000 and 1:600, SA-5594, Vector Laboratories) were applied to detect Iba-1. Brains from Aldh1l1^{GFP}, Il3^{GFPfl/fl}, Il3^{-/-} and WT mice were harvested and fixed in 4% paraformaldehyde solution at 4° C. overnight. After rinsing with PBS, the fixed brains were placed in 30% sucrose in PBS at 4° C. overnight. The brains were embedded in O.C.T. compound and serial frozen sections (10 μm) were prepared using CryoJane Tape Transfer System (Leica Biosystems). For Aldh1l1^{GFP} and Il3^{GFPfl/fl} mice, an anti-GFP antibody (1:400, ab13970, Abcam) and a goat anti-chicken IgY secondary antibody, Alexa Fluor 488 (1:100, A-11039, Thermo Fisher Scientific) were used to detect GFP-Aldh1l1 and GFP-IL3. An anti-IL3 antibody (1:5, 503902, MP2-8F8, BioLegend) followed by a biotinylated rabbit anti-rat IgG secondary antibody and streptavidin DyLight 594 (1:100, BA-4001 and 1:600, SA-5594, Vector Laboratories) were used for IL-3 detection in Aldh1l1^{GFP} mice. A GFAP, eFluor 615 monoclonal antibody (1:25, 42-9892-80, GA5, Thermo Fisher Scientific) was used to detect astrocytes on Il3^{GFP} mice. For co-localization of IL-3Ra with Iba1, an anti-Interleukin 3 Receptor Alpha antibody (1:50, 141039, US Biological) and an anti-Iba-1 antibody (1:50, ab5076, Abcam) were incubated at 4° C. overnight after blocking with 4% donkey serum in PBS. A donkey anti-rabbit IgG secondary antibody, Alexa Fluor 555 (1:100, A-31572, Thermo Fisher Scientific) and a donkey anti-goat IgG secondary antibody, Alexa Fluor 488 (1:100, A-11055, Thermo Fisher Scientific) were used to detect IL-3Ra and Iba-1 respectively. An Alexa Fluor 647 anti-β-Amyloid, 1-16 antibody (1:50, 803021, 6E10, BioLegend) was used to identify amyloid plaques in the brains. For Il3^{-/-} and WT mice, Doublecortin (1:400, 4604S, Cell Signaling Technology) and active Caspase-3 (1:50, 559565, C92-605, BD Biosciences) were stained to detect neuronal precursor cells and apoptotic cells respectively. A biotinylated goat anti-rabbit IgG secondary antibody and streptavidin DyLight 594 (1:100, BA-1000 and 1:600, SA-5594, Vector Laboratories) were used for the staining. Nuclei were counterstained with DAPI (1:3000, D21490, Thermo Fisher Scientific). The images were captured by using a digital scanner NanoZoomer 2.0RS (Hamamatsu, Japan) or an automated fluorescence microscope, BX63 (Olympus). Image analysis and quantification was done with ImageJ software. Microglia morphology analysis was done using the Skeletonize plug-in for ImageJ.

Mouse whole-mount 3-dimensional confocal imaging. Brains were excised from 5-month-old 5×FAD and Il3^{-/-}5×FAD animals, cut in half along the sagittal plane, and fixed in 4% paraformaldehyde for 24 hours at 4° C. Tissue was washed 3 times with PBS for 1 hour at room temperature then embedded in 4% agrose and 250 nm sections were cut using a Pelco 101 vibratome. Sections were washed 3 times in PBS containing 1% Triton-x100 for 30 minutes with gentle rotation and then incubated for 1 hour in blocking solution: PBS containing 1% Triton-x100, and 20% goat serum. Sections were then incubated for 3 days at 4° C. in anti-Iba1 (Wako) and Anti-β amyloid (already conjugated to AF488, Biolegend) primary antibodies each at a dilution of 1/300 in blocking solution. Sections were then washed 3 times in blocking solution followed by 3 washes in PBS containing 1% Triton-x100. Sections were incubated overnight at 4° C. in anti-rabbit AF633 (Life Technologies) at a dilution of 1/200 in blocking solution. Finally, sections were washed 3 times in PBS containing 1% Triton-x100. Prior to imaging, sections were cleared using RapiClear 1.49 by immersion in the clearing solution for 20 minutes at room temperature. The cleared tissues were then mounted on a custom-made sample holder and imaged using an Olympus FV1000 microscope. Images were processed with Amira 3D software.

Human. Brain paraffin-embedded slides were obtained from the Massachusetts Alzheimers Disease Research Center Brain Bank, and anti-IL-3 (1:100, 524379, US Biological), anti-IL-3Ra (1:50, 14-1239-82, 6H6, Thermo Fisher Scientific), Alex Fluor 488 anti-GFAP (1:50, 53-9892-82, GAS, Thermo Fisher Scientific), and anti-Iba-1 (1:200, 019-19741, FUJIFILM Wako Chemicals) were used as primary antibodies. Biotinylated goat anti-rabbit IgG and horse anti-mouse IgG secondary antibodies were applied for IL-3 and IL-3Ra respectively (1:100, BA-1000 and BA-2000, Vector Laboratories) followed by streptavidin DyLight 594 (1:600, SA-5594, Vector Laboratories). A goat anti-rabbit IgG secondary antibody, Alexa Fluor 488 (1:100, A-11034, Thermo Fisher Scientific) was used for Iba-1detection, and nuclei were counterstained with DAPI (1:3000, D21490, Thermo Fisher Scientific). All the slides were scanned by a digital scanner NanoZoomer 2.ORS (Hamamatsu, Japan).

Microglia density and spatial analysis. During a preprocessing stage the image quality of the images was improved by reducing the fluorescence bleed-through signal and increasing the signal contrast to optimize cellular segmentation. To enhance cell contrast we utilized a contrast-limited adaptive histogram equalization algorithm and spatially filtered and denoised the images. A watershed algorithm was implemented to count individual cells, and a threshold was determined to reject objects with an area less than 45 microns square. The center position was determined for each cell. For each individual fluorescence channel, all processing and threshold parameters were kept fixed across all samples to guarantee consistency in the segmentation process of both AB plaques and microglia. The spatial data analysis was performed in 2D on the obtained segmented cells using a KNN (k-nearest neighbor) algorithm from the scikit-learn python package. For each segmented AB plaque, we then calculated the total number of microglia present at different interval distances from the center of it. Specifically, we selected a fixed binning interval of 45 microns and incrementally calculated the number of microglia present in the circular area cantered on the AB plaque and in the ring-shaped regions bounded by the concentric circles of progressively incrementing radiuses. The number of counted microglia is then normalized by the area of the considered region divided by the total number of microglia present in the brain section, and the average microglia density is then plotted as a function of distance. All software was written in python utilizing opencv, numpy, and scikit-learn packages.

Molecular Biology. Enzyme-linked immunosorbent assay. Mouse. IL-3 levels were measured using enzyme-linked immunosorbent assay (ELISA) kit (Boster Biological) according to the manufacturer's instructions. 3 hours prior to sacrifice mice were injected with a biotinylated anti-IL-3 capture antibody (Biolegend) as previously described²⁹. Measurement of β-amyloid was done as previously described³⁹. Briefly, brains were extracted and corticies were directed and homogenized in 8 volumes of TBS containing 5 mM EDTA, phosphatase inhibitor (ThermoFisher), EDT-free protease inhibitor cocktail (Roche) and 2 mM 1,10-phenantroline (Sigma). Homogenates were centrifuged at 100,000 g for 1 hour at 4° C. using an Optima TL ultracentrifuge and a Ti70 rotor (Beckman Coulter). Supernatants were collected and used to measure TBS-soluble Aβ. The resulting pellet was homogenized in 70% formic acid. Samples were centrifuged at 100,000 g for 1 hour at 4° C. and supernatants were collected. Formic acid-containing supernatants were neutralized with 1M Tris-base, pH 11 (1:20 v:v) and samples were used to measure formic acid-soluble Aβ. Aβ40 and Aβ42 ELISAs were performed using Aβ ELISA kits (Wako). Human. Human brain IL-3 levels were measured using ELISA (Boster Biological). Briefly, samples of human cortex were weighed, homogenized in RIPA buffer and centrifuged at 8000 RPM for 2 minutes. IL-3 levels were measured in supernatant. Measurement of β-amyloid was done as previously described³⁹. Briefly, brains were extracted and corticies were directed and homogenized in 8 volumes of TBS containing 5 mM EDTA, phosphatase inhibitor (ThermoFisher), EDT-free protease inhibitor cocktail (Roche) and 2 mM 1,10-phenantroline (Sigma). Homogenates were centrifuged at 100,000 g for 1 hour at 4° C. using an Optima TL ultracentrifuge and a Ti70 rotor (Beckman Coulter). Supernatants were collected and used to measure TBS-soluble Aβ. The resulting pellet was homogenized in 70% formic acid. Samples were centrifuged at 100,000 g for 1 hour at 4° C. and supernatants were collected. Formic acid-containing supernatants were neutralized with 1M Tris-base, pH 11 (1:20 v:v) and samples were used to measure formic acid-soluble Aβ. Aβ40 and Aβ42 ELISAs were performed using Aβ ELISA kits (Wako).

Mouse qPCR. Total RNA was isolated using the RNeasy Mini Kit (Qiagen) or the NucleoSpin RNA XS kit (Takara Bio) according to the manufacturer's instructions. RNase-free DNase Set (Qiagen) was used for DNase digestion during RNA purification. RNA quantity and quality were assessed by Nanodrop for RNA isolated from tissues and with the Aglient RNA 6000 Pico kit (Aglient Technologies) on the Aglient 2100 Bioanalyzer for RNA of fluorescence-activated cell sorting (FACS)-purified cells. cDNA was generated from 1 μg of total RNA per sample using the High Capacity cDNA Reverse Transcription Kit (Applied Biosystems). Quantitative real-time TaqMan PCR was performed using the following FAM labelled TaqMan primers (Applied Biosystems): Il3 (Mm00439631_m1), Il3ra (Mm00434273_m1), Ccl2 (Mm00441242_m1), Complement C3 (Mm01232779_m1), Gfap (Mm01253033_m1), Ccl7 (Mm00443113_m1), Ccl5 (Mm01302427_m1), Il1β (Mm00434228_m1), Tnfa (Mm00443258_m1), Il6 (Mm00446190_m1), IL10 (Mm01288386_m1), Ccl12 (Mm01617100_m1), Trem2 (Mm04209424_g1), Syk (Mm01333032_m1), Tyrobp (Mm00449152_m1), Cd33 (Mm00491152_m1), Cd36 (Mm00432403_m1), Tlr4 (Mm00445273_m1), Sra (Mm00491755_m1), Cd206 (Mm01329362_m1), Mpp9 (Mm00442991_m1), Spp1 (Mm00436767_m1), Clec7a (Mm01183349_m1), Lyz2 (Mm04214174_uH), Apoe (Mm01307192_m1), Itgax (Mm00498708_g1), Itgam (Mm00434455_m1), Ptprc (Mm01293577_m1), Ctsg (Mm00456011_m1), Igf1 (Mm00439560), Cd68 (Mm03047343_m1). VIC labelled Actb (Mm00607939_s1) was used as the housekeeping gene. Results were analyzed by the comparative CT method. Average CT values for each sample were normalized to the average CT values of the housekeeping gene.

Human qPCR. RNA was extracted from human brain tissue (frontal cortex) with Trizol (Life Technologies) following manufacturer's instructions. The extracted RNA was dissolved in water and purified using the RNAeasy Mini Kit (Qiagen) according to the manufacturer's protocol. Alternatively, RNA was isolated using E.Z.N.A. Total RNA kit (Omega Blotek). Purified RNA was quantified using Qubit RNA Broad Range Assay Kit (Thermo Fisher Scientific) on the Qubit Fluorometer 3.0 (Thermo Fisher Scientific). RNA (1 μg) was reverse-transcribed using the SuperScript III First Strand Synthesis System and oligo-DT(20) primer (Invitrogen). Gene expression was assessed by performing Taqman real-time PCR assays. The probe targeting human Il3ra was labeled with FAM (Hs00608141_m1, Thermo Fisher Scientific). The probe targeting the human housekeeping gene Gapdh was labeled with VIC (Hs02786624_g1, Thermo Fisher Scientific). 1:10 diluted cDNAs were mixed with the probes and Taqman Universal Master Mix II (Applied Biosystems) and amplified using the C1000 Touch Thermal Cycler (Bio-Rad). Results were analyzed by the comparative CT method. Average CT values for each sample were normalized to the average CT values of the housekeeping gene. SNP genotyping. Genotyping was performed at two SNPs, rs429358 and rs7412, using a Taqman genotyping assay (Life Technologies) according to manufacturer's instructions.

Behavior phenotyping. Y-maze. Y-maze testing was adapted from published protocols⁴⁰. The Y-maze apparatus consisted of three arms joined in the middle to form a Y shape. The walls of the arms were 10 cm high and each were marked with a single large black letter serving as a spatial landmark and clue. With one arm of the maze closed, mice were allowed to explore the other two arms for 5 minutes before being returned to their home cage. Twenty minutes later, mice were returned to the Y-maze and allowed to explore all three arms for 5 min while being video recorded. The time spent in the new arm was quantified.

Morris Water Maze. The Morris Water Maze was conducted in the Animal Behavior Facility at the Massachusetts General Hospital. The Morris water maze test was performed with minor adjustment as previously described⁴¹. Spatial memory testing was conducted in a circular tank (diameter 1.22 m) filled with opacified water at 23° C. The water tank was dimly lit and surrounded by a white curtain. The maze was virtually divided into four quadrants, with one containing a hidden platform (diameter 10 cm) that was submerged 0.5 cm below the water level. Four prominent cues were placed outside the maze as spatial references. Mice were placed in the water facing the tank wall at different start positions across trials in a quasi-random fashion to prevent strategy learning. Mice were allowed to search for the platform for 1 minute; if the mice did not find the platform, they were guided toward it where they remained for 20 s. Each mouse went through four trials (one from each start position) per day for seven consecutive days. After each trial, the mouse was dried and placed back into its cage until the start of the next trial. All mouse movements were recorded by the computerized tracking system EthoVision XT (Noldus) that calculated distances moved and time required to reach the platform (escape latency), along with swim speed. The spatial probe trial was conducted 24 hours after the last training session (on day 8). For the probe trial, the platform was removed and mice were allowed to swim for 1 minute. The time spent by the mice in the area surrounding the location where the platform used to be (platform plus) was recorded. The platform plus surrounding the target is larger than the target itself, but smaller than the target quadrant. Data was calculated as time in the platform plus/60 s*100% and is given in percentage.

Illustrations. All illustrations were generated with a license to Biorender (biorender.com).

Statistics and reproducibility. Results are shown as mean±s.e.m. Statistical analysis was performed using GraphPad Prism 7 (Graphpad Software). Statistical tests included unpaired, two-tailed non-parametric Mann-Whitney U-tests (when Gaussian distribution was not assumed). For multiple comparisons, a non-parametric multiple-comparisons test comparing the mean rank of each group (when Gaussian distribution was not assumed) was used, or one- or two-way ANOVAs followed by Turkey's test were used. For correlation analysis the mean expression level from individuals of equal disease duration was determined and correlation was computed using Pearson correlation coefficients. P values of 0.05 or less were considered to denote significance. Each experiment was repeated independently at least 3 times with similar results.

TABLE 1
SpCas9 guide RNAs used to target Il3 and Il3ra.

| Gene | Spacer | Spacer | | |
|---|---|---|---|---|
| targeted | Description | Sequence | # | PAM |
| Il3 | mIl3-intron1-1 | GTAACTGGCT | 4 | TGG |
| | | GAGGTTGGCC | | |
| | mIl3-STOP-1 | TGAATGTTCC | 5 | TGG |
| | | TCATGGCCCA | | |
| Il3ra | mIl3ra-1 | GGGACCAATG | 6 | GGG |
| | | ATGTCACCTA | | |
| | mIl3ra-STOP-2 | AGACGCCTGA | 7 | GGG |
| | | GAACTGTGTG | | |

#, SEQ ID NO:

TABLE 2
Primers used to evaluate
genome editing efficiencies.

| Primer | Primer | | |
|---|---|---|---|
| ID | Sequence | # | Primer Description |
| oBK8657 | CTTGGAGGACCAGA | 8 | fwd amplicon primer to |
| | ACGAGACAATGG | | sequence intron 1 targets |
| | | | for mouse Il3 |
| oBK8658 | GAAGCAAGGCATCG | 9 | reverse amplicon primer |
| | TGGAGTGATGG | | to sequence intron 1 |
| | | | targets for mouse Il3 |
| oBK8660 | GTTTAGCAGGCTGT | 10 | fwd amplicon primer to |
| | GCCCTTGCCC | | sequence stop codon |
| | | | targets for mouse Il3 |
| oBK8663 | GACAAATGAACATG | 11 | reverse amplicon primer |
| | GCCCCAGTCTTCC | | to sequence stop codon |
| | | | targets for mouse Il3 |
| oBK8664 | GATGATGTCATTCT | 12 | fwd amplicon primer to |
| | CACCCCCAGATGTC | | sequence intron 1 targets |
| | | | for mouse Il3ra |
| oBK8667 | TGCAGGTTCTGGAT | 13 | reverse amplicon primer |
| | GGGCGTGGTC | | to sequence intron 1 |
| | | | targets for mouse Il3ra |
| oBK8668 | GGACAGGAAGTGAC | 14 | fwd amplicon primer to |
| | ACTGGGGGTCAG | | sequence stop codon |
| | | | targets for mouse Il3ra |
| oBK8671 | GCAATCCCTCTGTC | 15 | reverse amplicon primer |
| | TCAGCTCCTG | | to sequence stop codon |
| | | | targets for mouse Il3ra |

#, SEQ ID NO:

TABLE 3
Primers used to confirm insertion
of donor DNA by Sanger sequencing.

| Primer | Primer | | |
|---|---|---|---|
| ID | Sequence | # | Primer Description |
| oKAC227 | CCCTAGTG | 16 | fwd amplicon primer to |
| | TTTGCAGC | | sequence outside of left |
| | CATATCTC | | homology arm for mouse Il3 |
| | C | | |
| oKAC242 | CCAGGCCA | 17 | rev amplicon primer |
| | ACCATAAC | | overlapping left loxP site |
| | TTCGTATA | | to sequence mouse Il3 |
| | ATGTATG | | |
| oKAC229 | CTGTACAG | 18 | fwd amplicon primer in left |
| | TCAGGGTC | | homology arm to sequence |
| | AAGTTTGT | | mouse Il3 |
| | GC | | |
| oKAC225 | GGCGGATC | 19 | rev amplicon primer in |
| | TTGAAGTT | | EGFP to sequence mouse |
| | CACCTTGA | | Il3 |
| | TG | | |
| oKAC223 | GCTGACCC | 20 | fwd amplicon primer in |
| | TGAAGTTC | | EGFP to sequence mouse |
| | ATCTGC | | Il3 |
| oKAC231 | GCTCAGAT | 21 | rev amplicon primer outside |
| | GATGGTGG | | of right homology arm to |
| | TAGTGGAT | | sequence mouse Il3 |
| | AG | | |
| oKAC233 | GGACCATG | 22 | fwd amplicon primer to |
| | ACAGGAAC | | sequence outside of left |
| | CAGAAGC | | homology arm for mouse |
| | | | Il3ra |
| oKAC240 | GGCAAGTG | 23 | rev amplicon primer |
| | ACATGTCC | | overlapping left loxP site |
| | CTATAACT | | to sequence mouse Il3ra |
| | TCGTATAA | | |
| | TG | | |
| oBK8655 | CCCTAAGC | 24 | fwd amplicon primer in left |
| | TCTTCCCT | | homology arm to sequence |
| | TCTTGTTG | | mouse Il3ra |
| | GC | | |
| oBK8670 | CCTTCAGA | 25 | rev amplicon primer in right |
| | GCCCCACT | | homology arm to sequence |
| | TCCTGTCG | | mouse Il3ra |
| | AAG | | |
| oKAC224 | GACCACAT | 26 | fwd amplicon primer in |
| | GAAGCAGC | | EGFP to sequence mouse |
| | ACGACTTC | | Il3ra |
| oKAC237 | GTTACAAC | 27 | rev amplicon primer outside |
| | ACCTAGAA | | of right homology arm to |
| | GTAGTACC | | sequence mouse Il3ra |
| | TCCTC | | |

#, SEQ ID NO:

TABLE 4
Missense mutations detected in CRISPR Cas9 edited mice. Missense
mutations did not influence EGFP signal in Il3 targeted mice
but resulted in quenching of EGFP signaling in Il3rα
targeted mice. IL-3Rα functionality was not impacted.

| | Element of donor in | |
|---|---|---|
| Mouse | which missense | |
| line | mutation was detected | Missense mutations detected |
| Il3^{GFPfl/fl} | Il3 cDNA | D70Y (GAT > TAT) |
| | eGFP | E143D (GAG > GAT) |
| Il3rα^{fl/fl} | Il3ra cDNA | W295L (TGG > TTG) |
| | eGFP | G41C (GGC > TGC), E173Q |
| | | (GAG > CAG) and |
| | | A228S (GCC > TCC) |

TABLE 5
Primer sequences used for genotyping
Il3^{GFPfl/fl} and Il3^{rafl/fl} mice.

| | | Forward | Forward | Reverse | Reverse |
|---|---|---|---|---|---|
| Mouse | Primer | primer | primer | primer | primer |
| Line | pair | ID | sequence | ID | sequence |
| Il3^{GFPfl/fl} | 1 | oBK8660 | GTTTAGCAGGCTGTG | oKAC230 | CGCCAAGCCTGAATGA |
| | | | CCCTTGCCC | | AGTCCTAG |
| | | | (SEQ ID NO: 28) | | (SEQ ID NO: 29) |
| | 2 | oKAC224 | GACCACATGAAGCAG | oKAC230 | CGCCAAGCCTGAATGA |
| | | | CACGACTTC | | AGTCCTAG |
| | | | (SEQ ID NO: 30) | | (SEQ ID NO: 31) |
| Il3ra^{fl/fl} | 1 | oBK8668 | GGACAGGAAGTGACA | oKAC236 | CCAGAAGGAACCCGAG |
| | | | CTGGGGGTCAG | | CTTCATC |
| | | | (SEQ ID NO: 32) | | (SEQ ID NO: 33) |
| | 2 | oKAC224 | GACCACATGAAGCAG | oKAC236 | CCAGAAGGAACCCGAG |
| | | | CACGACTTC | | CTTCATC |
| | | | (SEQ ID NO: 34) | | (SEQ ID NO: 35) |

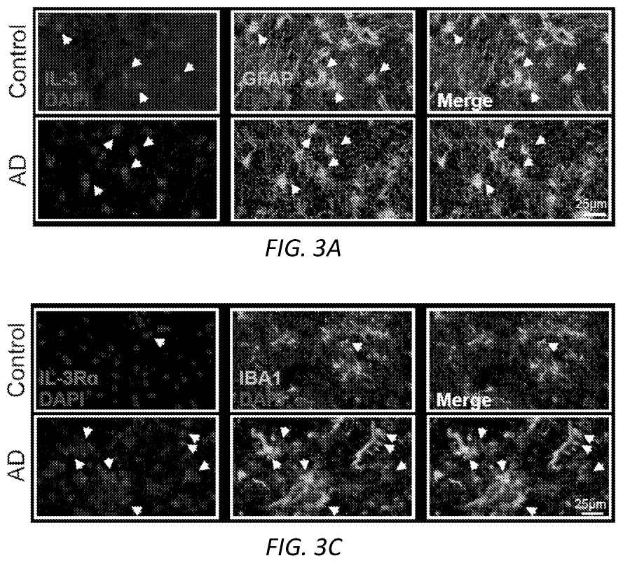

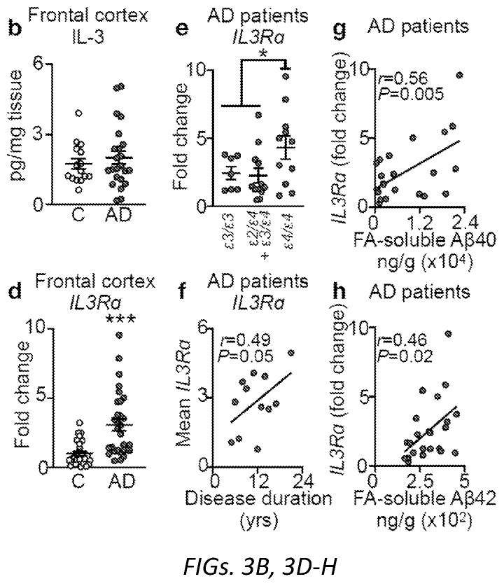

TABLE 6
Cohort Characteristics

| Characteristics of control and AD cases | | | Characteristics of control and AD cases | | | |
|---|---|---|---|---|---|---|
| used for FIGS. 3b, g, and h. | | | used for FIGS. 3d-f | | | |
| | Controls | | | Controls | | |
| Characteristics | (n = 15) | AD (n = 23) | Characteristics | (n = 28) | AD (n = 30) | |
| Age at death | 83.33 ± 10.28 | 74 ± 2.06 | Age at death | 81.44 ± 7.44 | 78.57 ± 10.73 | |
| (years) | | | (years) | | | |
| Disease | NA | 9.4 (5-12) | Disease | NA | 10.7 | (5-21) |

| | | | | | |
|---|---|---|---|---|---|
| duration | | | duration | | |
| (min-max) | | | (min-max) | | |
| Males/Females | 66.66/33.33 | 50/50 | Males/Females | 50/50 | 35.7/64.3 |
| (%) | | | (%) | | |

| | | | |
|---|---|---|---|
| APOEε4 carriers | 6 (3M/3F) | 23 | (9M/14F) |
| APOEε4 | 0 | 11 | (6M/5F) |

homozygous
carriers

### Example 1. Astrocytic Interleukin-3 Programs Microglia and Limits Alzheimer's Disease

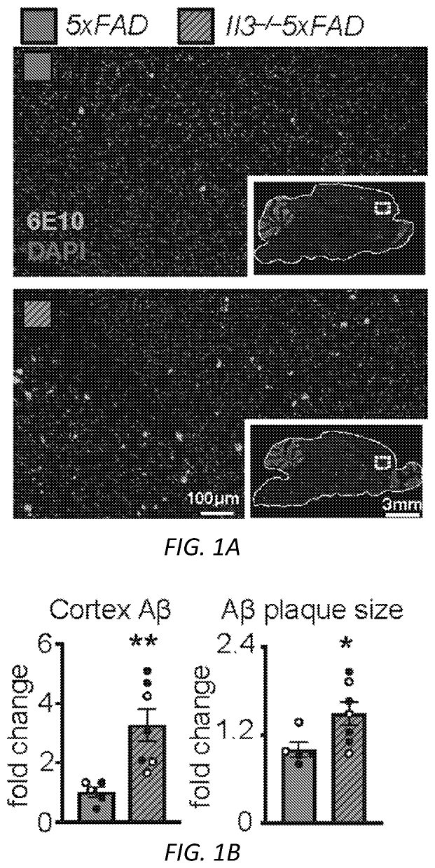

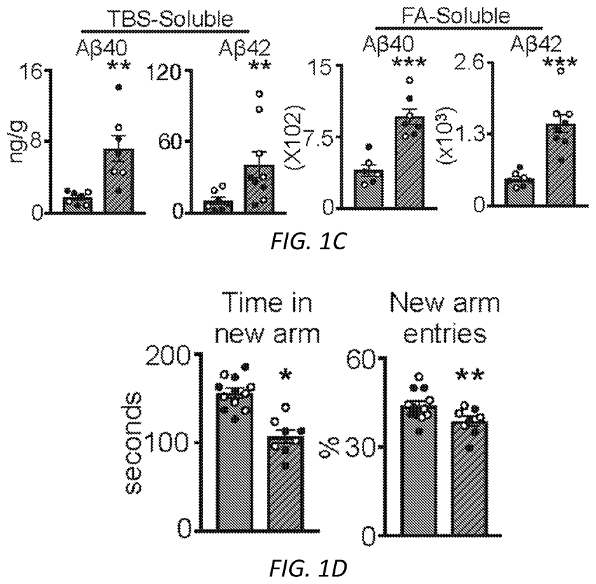

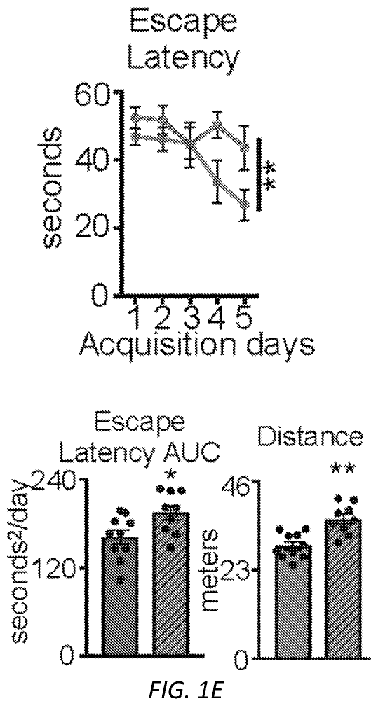

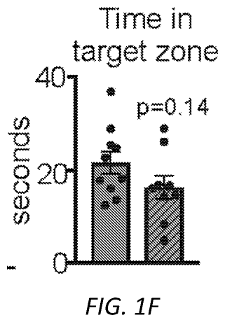

We profiled the brains of wildtype (WT) and IL-3-deficient (Il3^{-/-}) mice. In otherwise healthy animals, IL-3 deficiency did not impact blood brain barrier (BBB) permeability, neurogenesis, neuronal death, microglia activation and proliferation, or Y-maze memory. To test the function of IL-3 in AD, we crossed Il3^{-/-} mice with 5×FAD mice and found increased Aβ aggregates, Aβ plaque size, and Aβ levels in the cortex of Il3^{-/-}5×FAD mice (FIG. 1a-c). Il3^{-/-}5×FAD mice demonstrated impaired short-term (FIG. 1d) and spatial learning memory (FIG. 1e), and tended toward reduced memory retention (FIG. 1f). These observations suggest a protective role for IL-3 in a murine model of AD.

Because IL-3 governs haematopoiesis⁶, we assessed leukocyte generation. Compared to WT mice, 5×FAD mice had a higher number of haematopoietic progenitor cells in the bone marrow and more circulating myeloid cells. IL-3 deletion had modest effects on haematopoiesis in 5×FAD mice. Despite these findings, we did not observe altered BBB permeability or seeding of the brain parenchyma by peripheral leukocytes in 5×FAD or Il3^{-/-}5×FAD mice, suggesting that blood-derived leukocytes are rare in the 5×FAD brain, regardless of IL-3. While these data do not exclude vascular skull channels¹⁷ or the meninges¹⁸ as sources of immune cells^{19,20}, they do suggest that IL-3's function is in the local brain environment.

We measured IL-3 levels in plasma and the cerebrospinal fluid (CSF), and despite comparable levels in WT and 5×FAD mice, we noted a 4-fold increase in IL-3 concentration in the CSF relative to plasma, suggesting that IL-3 may be generated locally in the brain (FIG. 2a). To explore this possibility, we designed a CRISPR-Cas9-based editing strategy to generate dual Il3 reporter/floxed mice (Il3^{GFPfl/fl} mice, Tables 1-5). We confirmed successful sequence insertion and GFP signal in CD4⁺ T-cells, a known IL-3 source²¹. Strikingly, flow cytometry of whole brain tissue from Il3^{GFPfl/fl} mice revealed that a subset of astrocytes (˜4%), but not microglia or other CD45⁻ cells, produced IL-3 (FIG. 2b). Evaluation of Il3^{GFPfl/fl}5×FAD mice suggested that AD pathology does not appear to change astrocyte IL-3 production (FIG. 2b-c). Il3 expression was specific to astrocytes (FIG. 2d), age-dependent, and comparable between WT and 5×FAD mice. Imaging of Il3^{GFPfl/fl} mice showed co-localization of GFP with GFAP⁺ astrocytes (FIG. 2e), strengthening the idea that a subset of astrocytes are the primary source of IL-3 in the murine brain.

To investigate region-specific heterogeneity in astrocyte IL-3 production, we profiled astrocyte reporter (Aldh1l1^{GFP}) mice revealing co-localization of IL-3 with Aldh1l1-GFP⁺ astrocytes, but not Aldh1l1-GFP⁻ non-astrocytes (FIG. 2f). The proportion of IL-3⁺ astrocytes varied across brain structures (FIG. 2f). However, owing to IL-3's function as a secreted cytokine and its presence in the circulating CSF, tissue IL-3 levels were comparable throughout the brain. Astrocyte activation did not influence IL-3 production and IL-3 deletion did not alter astrocyte morphology or distribution in healthy or 5×FAD animals. Together, these results suggest that a subset of astrocytes constitutively generate IL-3.

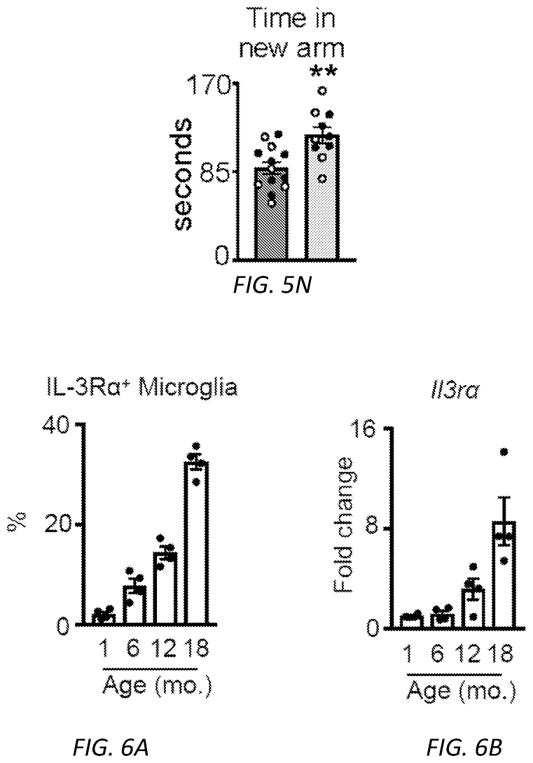

We sought to identify the brain cells that respond to IL-3. We uncovered an age-dependent increase of IL-3's specific receptor, IL-3Rα (also known as CD123), in microglia of WT mice (FIGS. 6a-b), but microglia of 5×FAD mice elevated IL-3Rα at a much earlier age (FIG. 2g-i). In 5- and 8-month-old 5×FAD mice, IL-3Rα⁺ microglia constituted more than 20% and 50% of the microglial pool, respectively. Comparatively, in WT mice IL-3Rα⁺ microglia accounted for ˜8% of cells at these ages. Premature IL-3Rα augmentation was specific to microglia and did not occur in other cell types of the brain (FIG. 2g) or in peripheral macrophages. Indeed, we confirmed robust IL-3Rα signaling in microglia from 5×FAD but not WT mice (FIG. 2j), and imaging demonstrated co-localization of IL-3Rα with microglia in close proximity to Aβ aggregates (FIG. 2k). These results indicate that microglia become responsive to IL-3 during AD by inciting IL-3Rα.

To explore networks that control dynamic Il3ra expression we profiled microglia of WT, Trem2^{-/-}, 5×FAD, and Trem2^{-/-}5×FAD mice by RNA-seq. TREM2 is an immuno-receptor that shapes the transcriptional and functional landscape of microglia²²⁻²⁴. We confirmed that Il3rα transcript is enriched in microglia of 5×FAD mice relative to WT mice at 4 (Log₂FC=2.068, p=3.45E⁻²⁷) and 8 (Log₂FC=2.851, p=1.12E⁻⁹³) months of age (FIG. 2l). We also noted an age-dependent increase in Il3rα (8- vs 4-month-old 5×FAD mice, Log₂FC=0.758, p=5.49E⁻⁰⁶). Strikingly, Trem2 deletion blunted Il3ra expression in 5×FAD mice at 4 (Log₂FC=−1.288, p=1.82E⁻⁰⁸) and 8 (Log₂FC=−1.552, p=1.60E⁻⁴⁰) months of age. Flow cytometry confirmed that Trem2 deletion abrogates the appearance of IL-3Rα⁺ microglia (FIG. 2m).

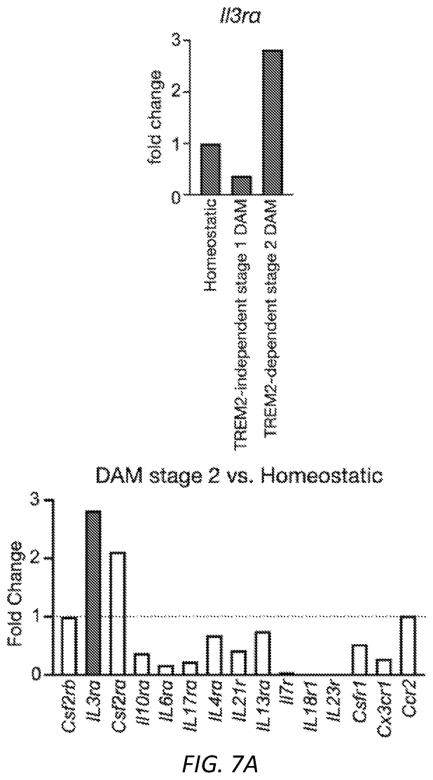

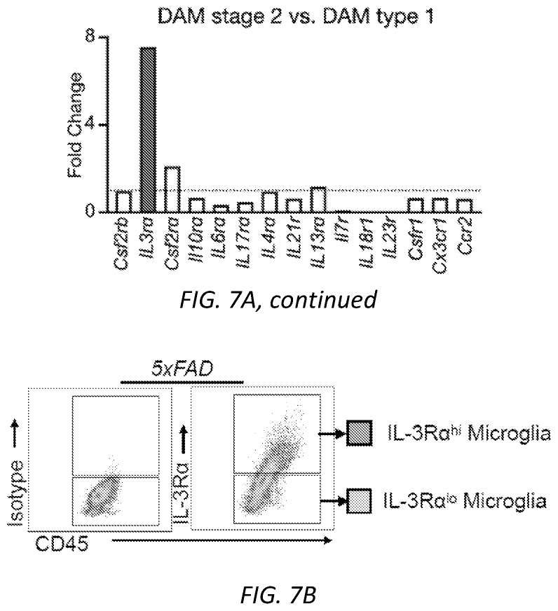

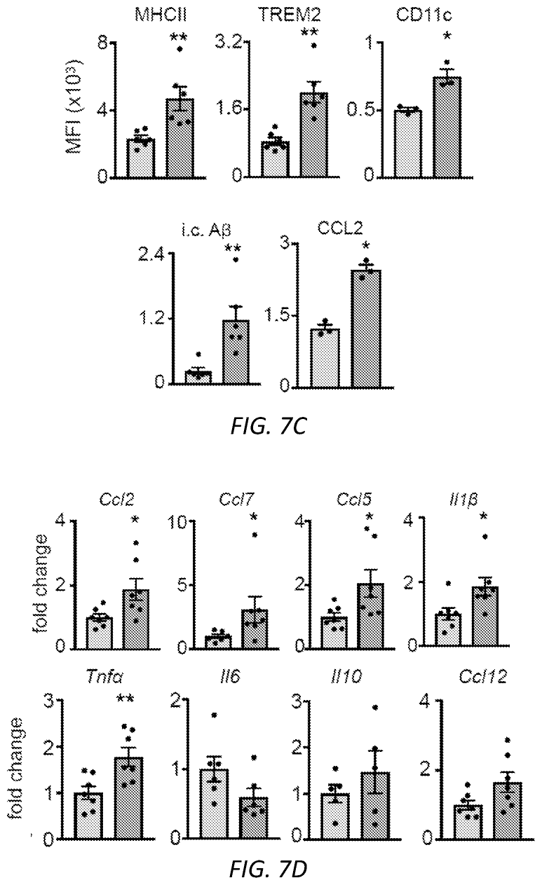

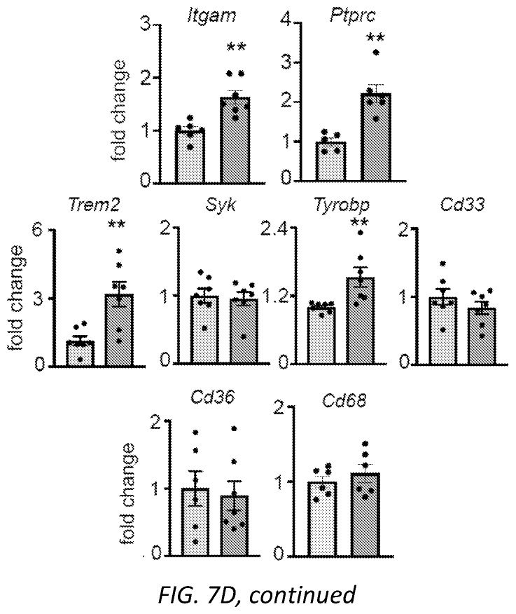

TREM2 mediates the development of disease associated microglia (DAM), an activated and protective phenotype occurring with AD^{22,25}. Analysis of public single-cell RNA-seq datasets²⁵ demonstrated that Il3ra is augmented in TREM2-dependent stage 2 DAMs, but not TREM2-independent stage 1 DAMs or homeostatic microglia (FIG. 7a). To investigate whether IL-3Rα⁺ microglia represent a phenotypically unique sub-population, we profiled IL-3Rα^{hi} and ^{lo} microglia in the brains of 5×FAD mice. In agreement with the hypothesis that TREM2 is required for IL-3Rα induction, IL-3Rα^{hi} microglia had higher levels of TREM2 and increased expression of the TREM2 adaptor protein DAP12 (Tyrobp). IL-3Rα^{hi} microglia also exhibited increased MHCII, CD11c (Itgax), CCL2, and intracellular Aβ, and more Ccl2, Ccl7, and Ccl5 expression. These findings point to IL-3Rα⁺ microglia as a distinct TREM2-dependent population endowed with an immune-responsive and activated phenotype.

Next, we assessed IL-3 signaling in the human brain (cohort characteristics in Table 6). Histology of postmortem frontal cortex from AD patients and age-matched non-demented controls uncovered IL-3 co-localizing with astrocytes (FIG. 3a). Measuring IL-3 protein in frontal cortex tissue homogenates suggested that IL-3 levels were unaltered by AD pathology (FIG. 3b). Meanwhile, microglia in the frontal cortex of healthy controls exhibited numerous thin ramifications, suggestive of a resting state, and were devoid of IL-3Rα (FIG. 3c). In contrast, microglia in AD patients stained for IL-3Rα abundantly and gained a globular and amoeboid morphology, indicative of microglial activation. Further, we observed a 3-fold increase in IL3Rα expression in the brain of AD patients (FIG. 3d), and patients carrying the AD-risk ε4/ε4 APOE genotype exhibited higher IL3Ra than carriers of other APOE genotypes (FIG. 3e). IL3Ra correlated with disease duration (FIG. 3f) and Aα levels in AD patients (FIG. 3g and h). Together, these findings suggest that AD pathology and severity drive microglia to express IL3Rα and indicate that IL-3 signaling is relevant in the human brain during AD pathogenesis.

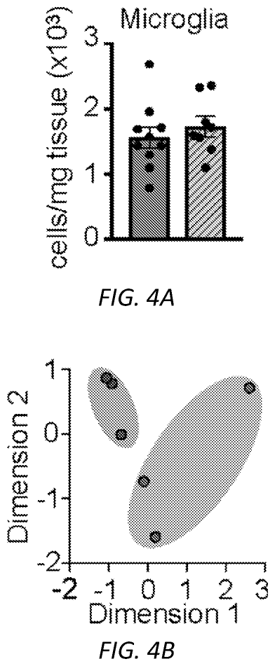

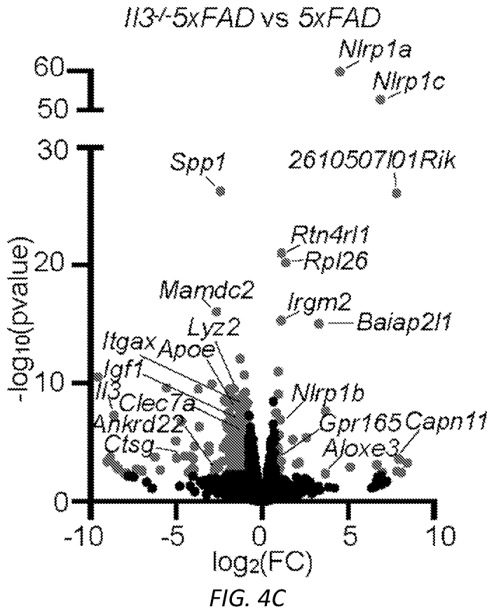

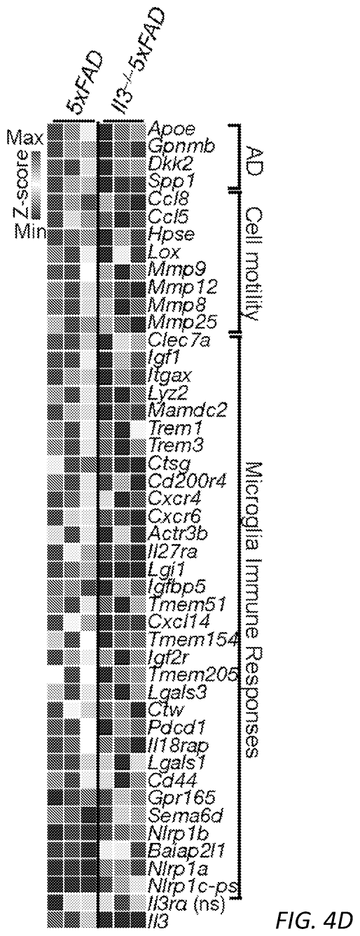

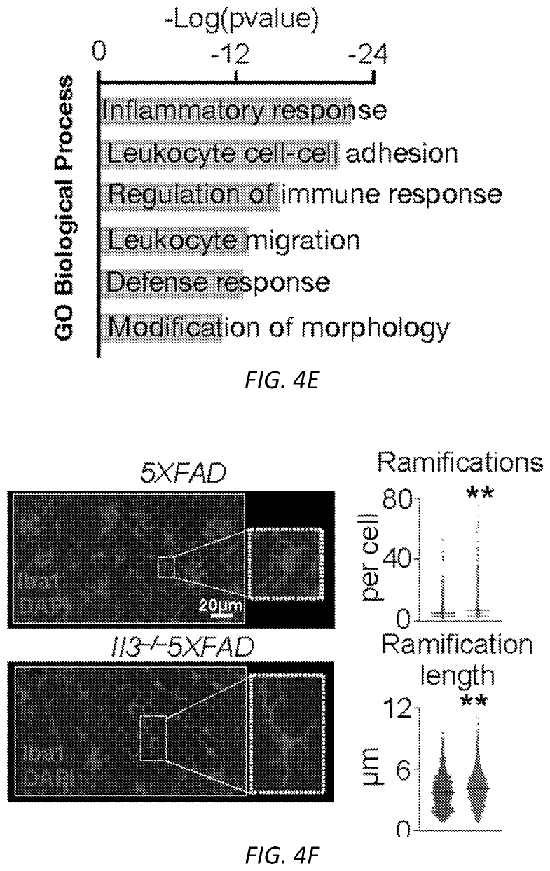

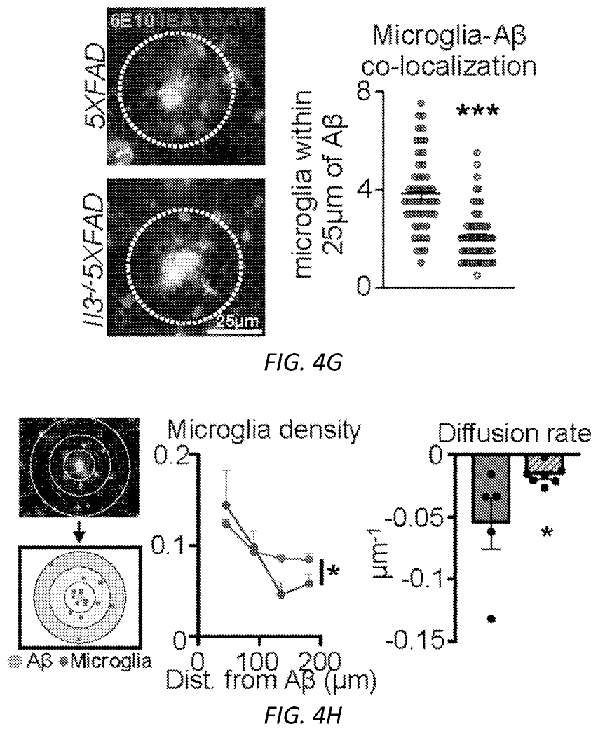

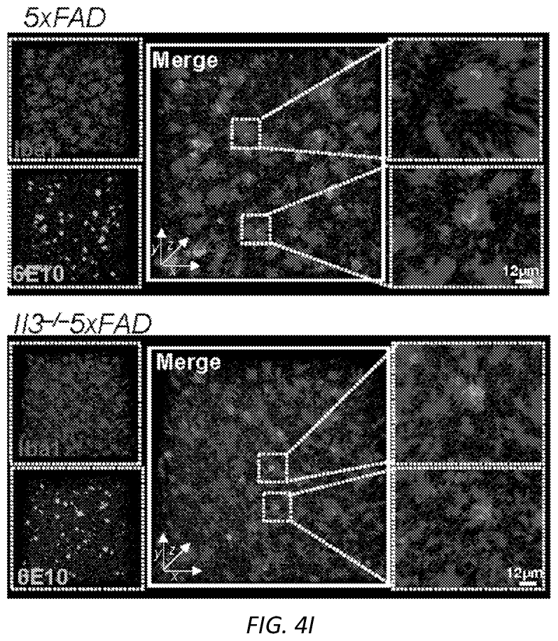

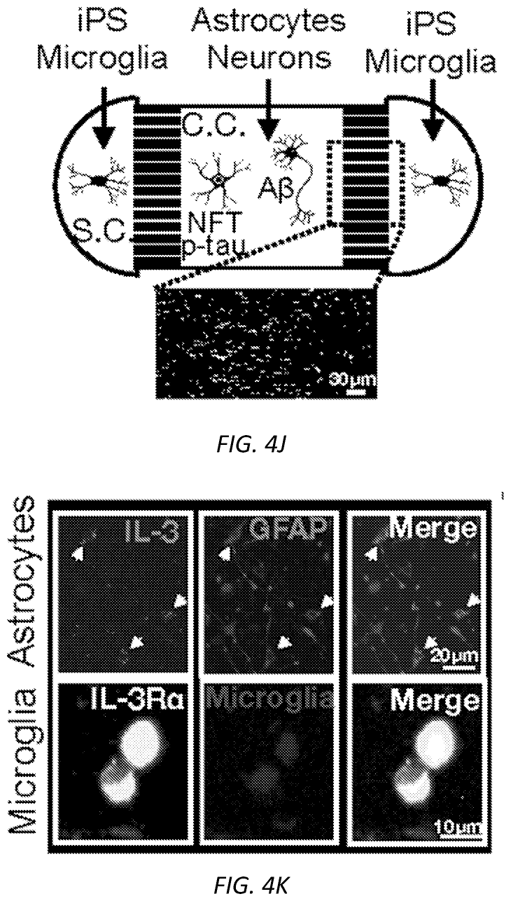

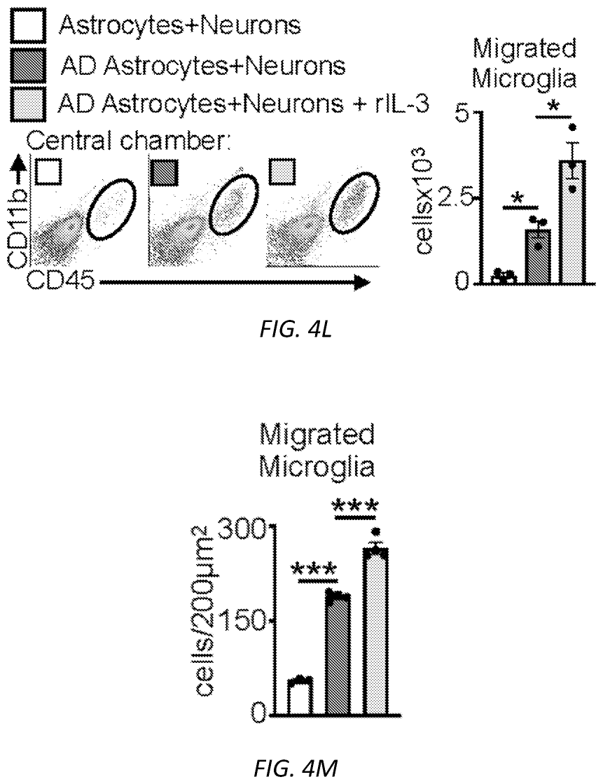

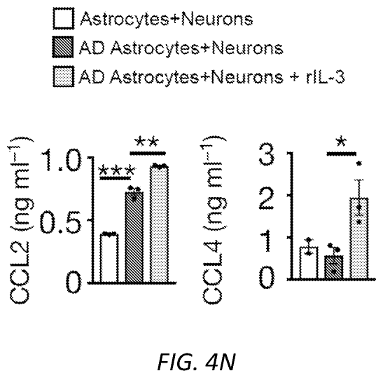

Having observed dynamic IL-3 signaling in AD, we explored IL-3's protective functions. In mice, IL-3 deletion did not influence microglia numbers (FIG. 4a) or proliferative capacity. We performed RNA-seq of microglia from 5×FAD and Il3^{-/-}5×FAD mice and identified 309 differentially expressed genes (269 decreased and 40 increased in Il3^{-/-}5×FAD vs 5×FAD, log₂FC>1.6, FDR<0.1, p<0.005) and a distinct transcriptional signature of Il3^{-/-}5×FAD microglia (FIG. 4b-c). Microglia from Il3^{-/-}5×FAD mice, despite an increased Aβ burden, had abrogated transcriptional activation of many genes indicative of AD and immune activation (FIG. 4c-d). Strikingly, IL-3 deletion led to reduced expression of Apoe, along with repressed expression of genes associated with AD and tissue repair (Spp1, Dkk2, Gpnmb), microglial immune responses (Clec7a, Igf1, Itgax, Lyz2, Mamdc2, Actr3b, Trem3, Trem1, Ctsg, Ctsw, Cd200r4, Clec4e, Cxcr4, Cxcr6, IL27ra), and genes critical to cell motility, extracellular matrix remodeling, and dissolution (Ccl8, Ccl5, Hpse, Lox, Mmp9, Mmp12, Mmp8, Mmp25). IL-3 regulated immune response, leukocyte migration, and modification of morphology pathways (FIG. 4e). Importantly, TREM2-dependent genes (e.g. Spp1, Itgax, Apoe, Lyz2, and Clec7a) were altered by IL-3 deletion, but Trem2 and Tyrobp were not (FC=0.8133, FDR=0.233 and FC=−0.43, FDR=0.09, respectively), bolstering the idea that IL-3 signaling acts downstream of TREM2. Collectively, these data demonstrate that IL-3 confers broad reprograming of the microglial transcriptome, deploying immune and motile responses.

Given our findings, we tested the role of IL-3 in shaping microglia morphology, motility, and distribution. Morphologic analysis revealed globular and rounded microglia in 5×FAD animals (FIG. 4f). Comparatively, microglia in Il3^{-/-}5×FAD mice exhibited a ramified morphology with numerous fine elongations, akin to a more homeostatic or resting state. IL-3-deficiency hindered microglial tissue mobilization and suppressed their ability to migrate towards and cluster around Aβ deposits (FIG. 4g). To expand on this observation, we used a watershed algorithm to compute the spatial orientation of microglia relative to Aβ plaque (FIG. 4h). As expected, in 5×FAD mice we observed high microglial density closest to Aβ which dissipated precipitously in concentric regions radiating from the plaque. In Il3^{-/-}5×FAD mice however, microglial density was more uniform resulting in a lower concentration of cells proximal to Aβ and a reduced rate of microglial diffusion. Building on these findings we performed three-dimensional (3D) whole-mount imaging of optically-cleared cortical tissue and chose areas with comparable Aβ burden to assess microglial morphology and spatial distribution. In 5×FAD mice, microglia were globular and clustered Aβ (FIG. 4i). In Il3^{-/-}5×FAD mice microglia were more ramified, disperse, and uniformly distributed, and their ability to cluster Aβ was impaired. The ability of microglia to phagocytose Aβ was independent of IL-3 as we did not observe changes in the expression of machinery important to Aβ phagocytosis (e.g. Axl, Dcstamp, Mertk, Cd36, Cd47, Msra), or the ability of microglia to ingest Aβ. Additionally, IL-3 did not influence the production of inflammatory cytokines (Ifnγ, Il18, Il1β, Il6, Tnfa). These findings suggest a specific role for IL-3 in instigating microglial immune activation, parenchymal re-distribution, and clustering around Aβ aggregates, which facilitate a microglial barrier and Aβ clearance prior to the establishment of unresolving tissue-damaging inflammation.

To explore the capacity of IL-3 to mediate the motility of human microglia directly, we used a 3D microfluidic triculture system that mimics the in vivo human AD environment^{26,27} (FIG. 4j). The central chamber of the microfluidic system was loaded with human GFP⁺ neurons and astrocytes differentiated from either control progenitor cells or AD cells that over-express mutated Aβ precursor protein. In the side chambers, we plated labeled human induced pluripotent stem cell (iPS)-derived adult microglia. The central and side chambers are linked by migration channels. First, we confirmed the presence of mature neurons, astrocytes, and neurofibrillary p-tau tangles along with augmented Aβ in central chambers plated with AD cells. Similar to our observations in murine and human brains, we observed co-localization of IL-3 with astrocytes and IL-3Rα with iPS microglia (FIG. 4k). Addition of rIL-3 to the AD tricultures robustly increased microglia migration to the central chamber (FIG. 4l-m) and augmented CCL2 and CCL4 levels (FIG. 4n-o). Reflecting our in vivo data, these results point to a critical role for IL-3 in microglial recruitment towards human Aβ aggregates and neurofibrillary p-tau.

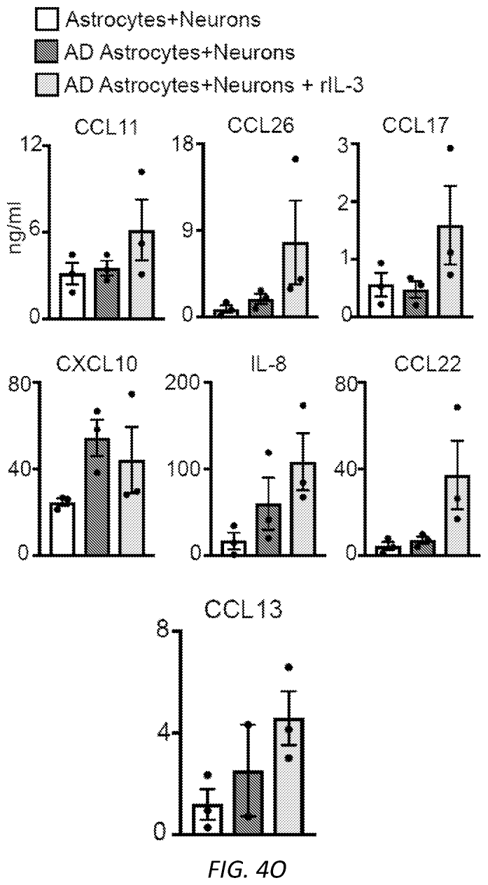

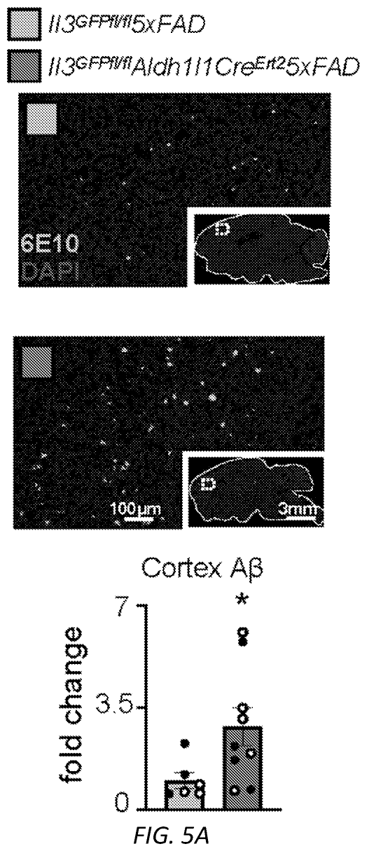

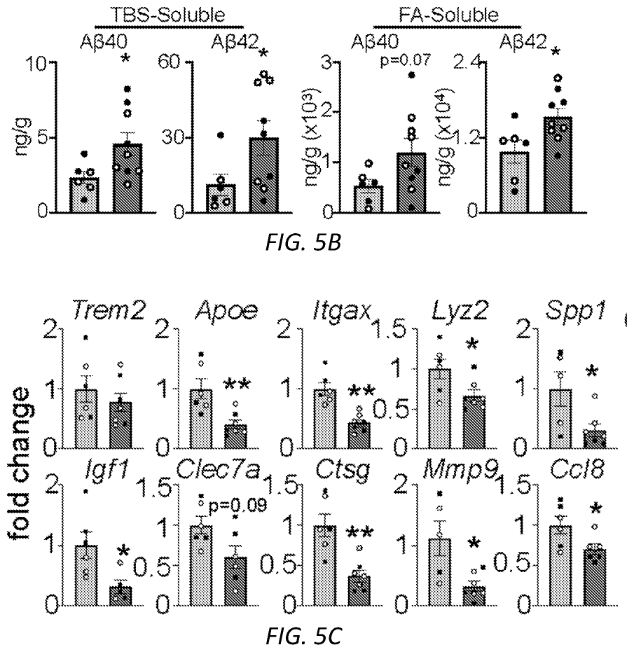

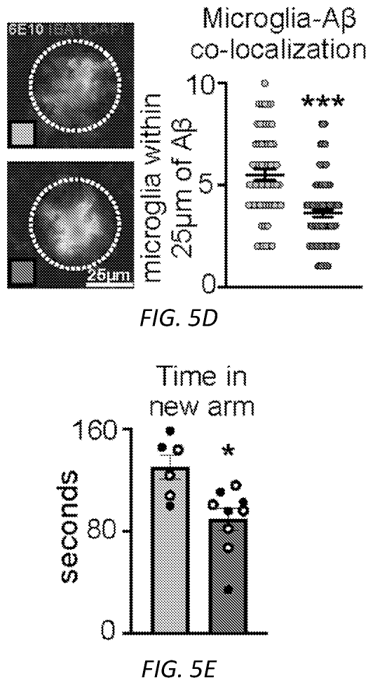

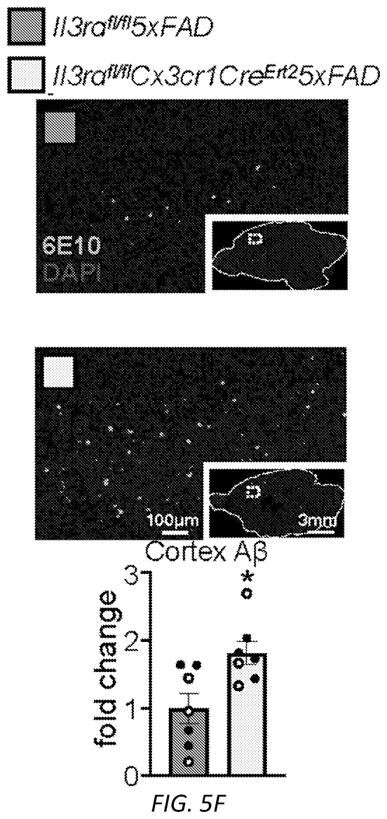

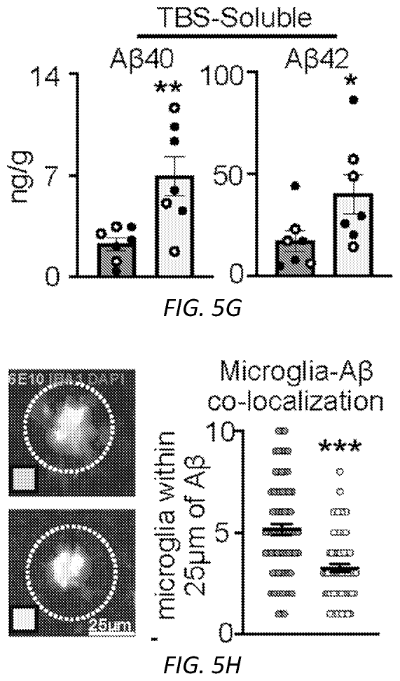

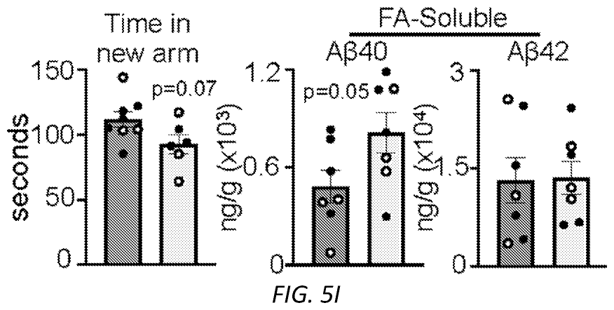

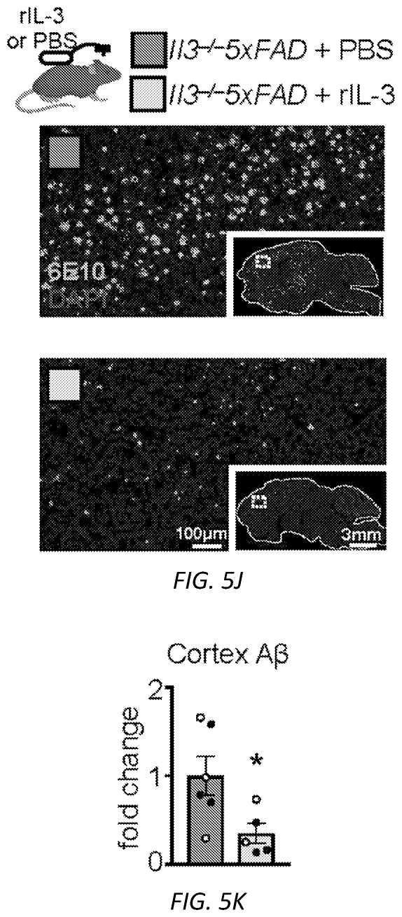

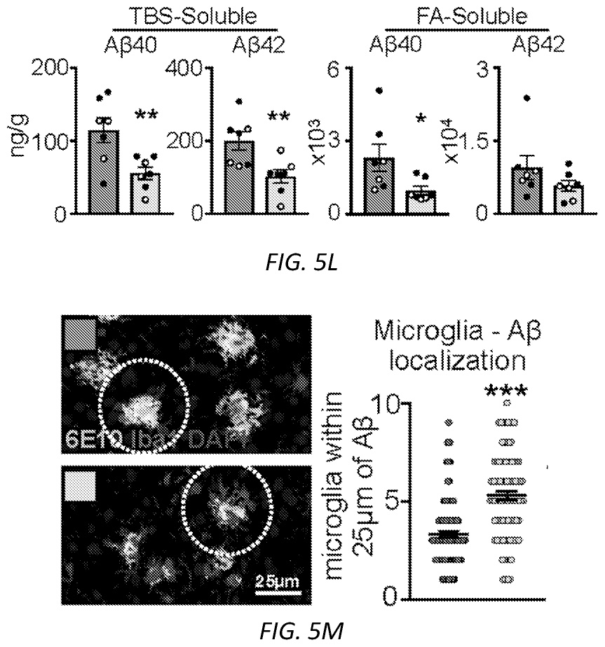

Next, we sought to determine the specific contribution of astrocyte IL-3 and microglia IL-3Rα to AD. We generated inducible astrocyte-specific Il3 knockout 5×FAD mice (Il3^{GFPfl/fl}Aldh1l1Cre^{ERT2}5×FAD) and repetitively injected them with tamoxifen which ablated astrocyte IL-3 production and reduced CSF IL-3 levels by 75%. Deletion of astrocyte-sourced IL-3 resulted in greater Aβ deposition (FIGS. 5a-b), repressed microglia expression of Apoe, Itgax, Lyz2, Spp1, Igf1, Clec7a, Ctsg, Mmp9, and Ccl8, but not Trem2 (FIG. 5c), and limited microglial clustering of Aβ (FIG. 5d). Memory was also impaired (FIG. 5e). To target microglial IL-3Rα, we employed a similar CRISPR-Cas9 editing strategy to generate mice with loxp sequences flanking Il3ra (Tables 1-5). We generated inducible microglia-specific Il3ra knockout 5×FAD mice (Il3ra^{fl/fl}Cx3cr1Cre^{ERT2}5×FAD) and repetitively injected them with tamoxifen abrogating the appearance of IL-3Rα⁺ microglia. Relative to controls, Il3ra^{fl/fl}Cx3cr1Cre^{ERT2}5×FAD mice had greater Aβ levels (FIGS. 5f-g), reduced microglia-Aβ co-localization (FIG. 5h), and tended towards worsened memory (FIG. 5i). Using cell-specific approaches, these findings propose IL-3-mediated astrocyte-microglia communication as a critical regulator of microglia reprograming that protects against AD pathology.

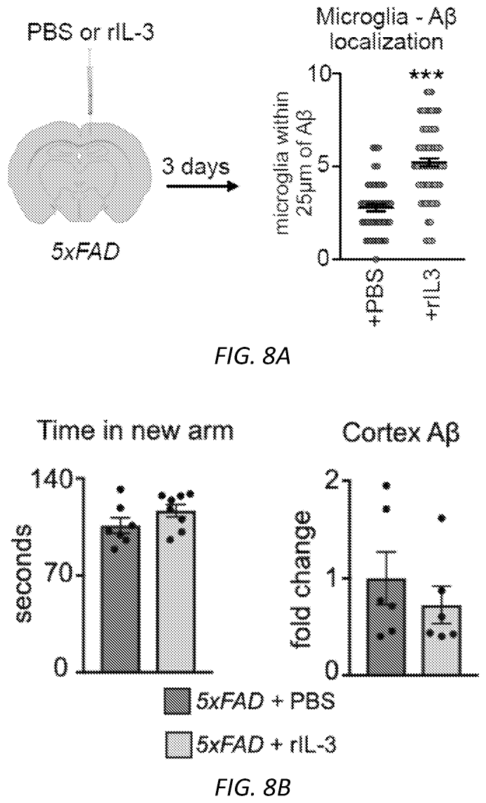

These results raised the possibility of utilizing IL-3 therapeutically. To explore this, we stereotactically injected rIL-3 into the cortex of 5×FAD animals. This led to a robust and rapid (<3 day) mobilization of microglia and clustering around Aβ (FIG. 8a). We extended this observation by delivering continuous rIL-3 into the lateral ventricle for 28 days which reduced Aβ load (FIGS. 5j-l). While microglia numbers were unaltered by rIL-3 infusion (3.4±0.4×10³ vs. 2.9±0.6×10³ cells/mg, PBS vs. rIL-3 infusion), clustering of Aβ deposits increased (FIG. 5m) and memory improved (FIG. 5n). The location of delivery was critical as 10 weeks of peripheral rIL-3 injections (i.p. rIL-3 or PBS, 10 ug 2×/week, i.p.) did not influence AD pathology (FIG. 8b). Collectively, these data support therapeutic use of IL-3 in AD.

### REFERENCES

1. Linnerbauer, M., Wheeler, M. A. & Quintana, F. J. Astrocyte Crosstalk in CNS Inflammation. Neuron 1-15 (2020) doi:10.1016/j.neuron.2020.08.012.

2. Vainchtein, I. D. & Molofsky, A. V. Astrocytes and Microglia: In Sickness and in Health. Trends Neurosci. 43, 144-154 (2020).

3. Castellani, G. & Schwartz, M. Immunological Features of Non-neuronal Brain Cells: Implications for Alzheimer's Disease Immunotherapy. Trends in Immunology (2020) doi:10.1016/j.it.2020.07.005.

4. Fakhoury, M. Microglia and astrocytes in Alzheimer's disease: implications for therapy. Curr. Neuropharmacol. (2017) doi:10.2174/1570159x15666170720095240.

5. Long, J. M. & Holtzman, D. M. Alzheimer Disease: An Update on Pathobiology and Treatment Strategies. Cell (2019) doi:10.1016/j.cell.2019.09.001.

6. Mindur, J. E. & Swirski, F. K. Growth factors as immunotherapeutic targets in cardiovascular disease. Arterioscler. Thromb. Vasc. Biol. 39, 1275-1287 (2019).

7. Ravetti, M. G. & Moscato, P. Identification of a 5-protein biomarker molecular signature for predicting Alzheimer's disease. PLoS One (2008) doi:10.1371/journal.pone.0003111.

8. Ray, S. et al. Classification and prediction of clinical Alzheimer's diagnosis based on plasma signaling proteins. Nat. Med. (2007) doi:10.1038/nm1653.

9. Britschgi, M. et al. Modeling of pathological traits in Alzheimer's disease based on systemic extracellular signaling proteome. Mol. Cell. Proteomics 10, 1-11 (2011).

10. Soares, H. D. et al. Plasma biomarkers associated with the apolipoprotein E genotype and alzheimer disease. Arch. Neural. 69, 1310-1317 (2012).

11. Huberman, M. et al. Correlation of cytokine secretion by mononuclear cells of Alzheimer patients and their disease stage. J. Neuroimmunol. (1994) doi:10.1016/0165-5728(94)90108-2.

12. Kiddle, S. J. et al. Plasma Based Markers of [11C] PiB-PET Brain Amyloid Burden. PLoS One (2012) doi:10.1371/journal.pone.0044260.

13. Frei, K., Bodmer, S., Schwerdel, C. & Fontana, A. Astrocytes of the brain synthesize interleukin 3-like factors. J. Immunol. (1985).

14. Frei, K., Bodmer, S., Schwerdel, C. & Fontana, A. Astrocyte-derived interleukin 3 as a growth factor for microglia cells and peritoneal macrophages. J. Immunol. (1986).

15. Zambrano, A., Otth, C., B. Maccioni, R. & I. Concha, I. IL-3 Control Tau Modifications and Protects Cortical Neurons from Neurodegeneration. Curr. Alzheimer Res. (2010) doi:10.2174/156720510793499011.

16. Zambrano, A., Otth, C., Mujica, L., Concha, I. I. & Maccioni, R. B. Interleukin-3 prevents neuronal death induced by amyloid peptide. BMC Neurosci. (2007) doi:10.1186/1471-2202-8-82.

17. Herisson, F. et al. Direct vascular channels connect skull bone marrow and the brain surface enabling myeloid cell migration. Nat. Neurosci. (2018) doi:10.1038/s41593-018-0213-2.

18. Gate, D. et al. Clonally expanded CD8 T cells patrol the cerebrospinal fluid in Alzheimer's disease. Nature (2020) doi:10.1038/s41586-019-1895-7.

19. Zenaro, E. et al. Neutrophils promote Alzheimer's disease-like pathology and cognitive decline via LFA-1 integrin. Nat. Med. (2015) doi:10.1038/nm.3913.

20. Pasciuto, E. et al. Microglia Require CD4 T Cells to Complete the Fetal-to-Adult Transition. Cell 182, 625-640.e24 (2020).

21. Anzai, A. et al. Self-reactive CD4+ IL-3+ T cells amplify autoimmune inflammation in myocarditis by inciting monocyte chemotaxis. J. Exp. Med. 216, 369-383 (2019).

22. Zhou, Y. et al. Human and mouse single-nucleus transcriptomics reveal TREM2-dependent and TREM2-independent cellular responses in Alzheimer's disease. Nat. Med. 26, 131-142 (2020).

23. Keren-Shaul, H. et al. A Unique Microglia Type Associated with Restricting Development of Alzheimer's Disease. Cell (2017) doi:10.1016/j.cell.2017.05.018.

24. Griciuc, A. et al. TREM2 Acts Downstream of CD33 in Modulating Microglial Pathology in Alzheimer's Disease. Neuron 103, 820-835.e7 (2019).

25. Keren-Shaul, H. et al. A Unique Microglia Type Associated with Restricting Development of Alzheimer's Disease. Cell 169, 1276-1290.e17 (2017).

26. Choi, S. H. et al. A three-dimensional human neural cell culture model of Alzheimer's disease. Nature 515, 274-278 (2014).

27. Park, J. et al. A 3D human triculture system modeling neurodegeneration and neuroinflammation in Alzheimer's disease. Nat. Neurosci. 21, 941-951 (2018).

28. Oakley, H. et al. Intraneuronal β-amyloid aggregates, neurodegeneration, and neuron loss in transgenic mice with five familial Alzheimer's disease mutations: Potential factors in amyloid plaque formation. J. Neurosci. (2006) doi:10.1523/JNEUROSCI.1202-06.2006.

29. Weber, G. F. et al. Interleukin-3 amplifies acute inflammation and is a potential therapeutic target in sepsis. Science (80-.). 347, 1260-1265 (2015).

30. Turnbull, I. R. et al. Cutting Edge: TREM-2 Attenuates Macrophage Activation. J. Immunol. (2006) doi:10.4049/jimmunol.177.6.3520.

31. Doench, J. G. et al. Optimized sgRNA design to maximize activity and minimize off-target effects of CRISPR-Cas9. Nat. Biotechnol. (2016) doi:10.1038/nbt.3437.

32. Bae, S., Park, J. & Kim, J. S. Cas-OFFinder: A fast and versatile algorithm that searches for potential off-target sites of Cas9 RNA-guided endonucleases. Bioinformatics (2014) doi:10.1093/bioinformatics/btu048.

33. Hsiau, T. et al. Inference of CRISPR Edits from Sanger Trace Data. bioRxiv (2018) doi:10.1101/251082.

34. Kleinstiver, B. P. et al. Engineered CRISPR-Cas12a variants with increased activities and improved targeting ranges for gene, epigenetic and base editing. Nat. Biotechnol. (2019) doi:10.1038/s41587-018-0011-0.

35. Rohland, N. & Reich, D. Cost-effective, high-throughput DNA sequencing libraries for multiplexed target capture. Genome Res. (2012) doi:10.1101/gr.128124.111.

36. Robbins, C. S. et al. Local proliferation dominates lesional macrophage accumulation in atherosclerosis. Nat. Med. (2013) doi:10.1038/nm.3258.

37. DeVos, S. L. & Miller, T. M. Direct intraventricular delivery of drugs to the rodent central nervous system. J. Vis. Exp. (2013) doi:10.3791/50326.

38. McQuade, A. et al. Development and validation of a simplified method to generate human microglia from pluripotent stem cells. Mol. Neurodegener. (2018) doi:10.1186/s13024-018-0297-x.

39. Griciuc, A. et al. Alzheimer's disease risk gene cd33 inhibits microglial uptake of amyloid beta. Neuron 78, 631-643 (2013).

40. Kraeuter, A. K., Guest, P. C. & Sarnyai, Z. The Y-Maze for Assessment of Spatial Working and Reference Memory in Mice. in Methods in Molecular Biology (2019). doi:10.1007/978-1-4939-8994-2_10.

41. Vorhees, C. V. & Williams, M. T. Morris water maze: Procedures for assessing spatial and related forms of learning and memory. Nat. Protoc. (2006) doi:10.1038/nprot.2006.116.

42. Luo X-j, Li M, Huang L, Nho K, Deng M, Chen Q, et al. (2012) The Interleukin 3 Gene (IL3) Contributes to Human Brain Volume Variation by Regulating Proliferation and Survival of Neural Progenitors. PLoS ONE 7(11): e50375.

43. Christine Chavany, Carlos Vicario-Abejón, Georgina Miller, Moncef Jendoubi. Transgenic mice for interleukin 3 develop motor neuron degeneration associated with autoimmune reaction against spinal cord motor neurons. Proceedings of the National Academy of Sciences Sep 1998, 95 (19) 11354-11359; DOI: 10.1073/pnas.95.19.11354.

44. Calsolaro, V. and Edison, P. (2016), Neuroinflammation in Alzheimer's disease: Current evidence and future directions. Alzheimer's & Dementia, 12: 719-732.

45. US2017/0189490

46. Sarlus H, Heneka MT. Microglia in Alzheimer's disease. J Clin Invest. 2017;127(9):3240-3249. doi:10.1172/JCI90606

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject with Alzheimer's disease, the method comprising administering a therapeutically effective amount of an Interleukin 3 Receptor (IL3R) agonist.

2. The method of claim 1, wherein the IL3R agonist is (i) an IL3 peptide or an IL3R polypeptide; or (ii) a nucleic acid encoding an IL3 peptide or a nucleic acid encoding an IL3R peptide.

3. The method of claim 2, wherein the nucleic acid encoding an IL3 peptide or an IL3R polypeptide comprises mRNA.

4. The method of claim 2, wherein the nucleic acid encoding an IL3 peptide or an IL3R polypeptide is in an expression vector.

5. The method of claim 4, wherein the expression vector comprises a nucleic acid encoding an IL3 peptide and a promotor that directs expression of the IL3 peptide in astrocytes, optionally a GFAP or Aldh1l1 promoter.

6. The method of claim 4, wherein the expression vector comprises a nucleic acid encoding an IL3R polypeptide and a promoter that directs expression of the IL3R polypeptide in microglia, optionally a CD11b or Iba1 promoter.

7. The method of claim 4, wherein the expression vector is a viral vector.

8. The method of claim 7, wherein the viral vector is an adeno-associated virus (AAV) vector.

9. The method of claim 8, wherein the AAV is selected from the group consisting of AAV9, AAV-F, AAV1, AAV2, AAV3, AAV4, AAV5, AAV6, AAV7, AAV8, AAV2/1, AAV2/2, AAV2/5, AAV2/6, AAV2/7, AAV2/8, AAVrh10, AAV11, and AAV12.

10. The method of claim 1, wherein the IL3R agonist is administered in a microvesicle.

11. The method of claim 10, wherein the microvesicle comprises a nucleic acid encoding an IL3 peptide and a promotor that directs expression of the IL3 peptide in astrocytes, optionally GFAP or Aldh1l1, and/or a nucleic acid encoding an IL3R polypeptide and a promoter that directs expression of the IL3R polypeptide in microglia, optionally CD11b or Iba1.

12. The method of claim 1, wherein the IL3R agonist is administered into the CNS via infusion or injection into the cerebrospinal fluid (CSF), intrathecally, or by direct injection or infusion using stereotactic methods.

13.-24. (canceled)


SEQUENCE LISTING (35 sequences)

SEQ ID NO: 1 - protein, 152 aa, Homo sapiens
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 2 - protein, 112 aa, Artificial Sequence
  Description of Artificial Sequence Synthetic polypeptide
ANCSIMIDEIIHHLKRPPNPLLDPNNLNSEDMDILMERNLRTPNLLAFVRAVKHLENASGIEAILRNLQPCLPSATAAPSRHPIIIKAGDWQEFREKLTFYLVTLEQAQEQQ

SEQ ID NO: 3 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 4 - DNA, 20 bp, Artificial Sequence
  Description of Artificial Sequence Synthetic oligonucleotide
gtaactggctgaggttggcc

SEQ ID NO: 5 - DNA, 20 bp, Artificial Sequence
  Description of Artificial Sequence Synthetic oligonucleotide
tgaatgttcctcatggccca

SEQ ID NO: 6 - DNA, 20 bp, Artificial Sequence
  Description of Artificial Sequence Synthetic oligonucleotide
gggaccaatgatgtcaccta

SEQ ID NO: 7 - DNA, 20 bp, Artificial Sequence
  Description of Artificial Sequence Synthetic oligonucleotide
agacgcctgagaactgtgtg

SEQ ID NO: 8 - DNA, 26 bp, Artificial Sequence
  Description of Artificial Sequence Synthetic primer
cttggaggaccagaacgagacaatgg

SEQ ID NO: 9 - DNA, 25 bp, Artificial Sequence
  Description of Artificial Sequence Synthetic primer
gaagcaaggcatcgtggagtgatgg

SEQ ID NO: 10 - DNA, 24 bp, Artificial Sequence
  Description of Artificial Sequence Synthetic primer
gtttagcaggctgtgcccttgccc

SEQ ID NO: 11 - DNA, 27 bp, Artificial Sequence
  Description of Artificial Sequence Synthetic primer
gacaaatgaacatggccccagtcttcc

SEQ ID NO: 12 - DNA, 28 bp, Artificial Sequence
  Description of Artificial Sequence Synthetic primer
gatgatgtcattctcacccccagatgtc

SEQ ID NO: 13 - DNA, 24 bp, Artificial Sequence
  Description of Artificial Sequence Synthetic primer
tgcaggttctggatgggcgtggtc

SEQ ID NO: 14 - DNA, 26 bp, Artificial Sequence
  Description of Artificial Sequence Synthetic primer
ggacaggaagtgacactgggggtcag

SEQ ID NO: 15 - DNA, 24 bp, Artificial Sequence
  Description of Artificial Sequence Synthetic primer
gcaatccctctgtctcagctcctg

SEQ ID NO: 16 - DNA, 25 bp, Artificial Sequence
  Description of Artificial Sequence Synthetic primer
ccctagtgtttgcagccatatctcc

SEQ ID NO: 17 - DNA, 31 bp, Artificial Sequence
  Description of Artificial Sequence Synthetic primer
ccaggccaaccataacttcgtataatgtatg

SEQ ID NO: 18 - DNA, 26 bp, Artificial Sequence
  Description of Artificial Sequence Synthetic primer
ctgtacagtcagggtcaagtttgtgc

SEQ ID NO: 19 - DNA, 26 bp, Artificial Sequence
  Description of Artificial Sequence Synthetic primer
ggcggatcttgaagttcaccttgatg

SEQ ID NO: 20 - DNA, 22 bp, Artificial Sequence
  Description of Artificial Sequence Synthetic primer
gctgaccctgaagttcatctgc

SEQ ID NO: 21 - DNA, 26 bp, Artificial Sequence
  Description of Artificial Sequence Synthetic primer
gctcagatgatggtggtagtggatag

SEQ ID NO: 22 - DNA, 23 bp, Artificial Sequence
  Description of Artificial Sequence Synthetic primer
ggaccatgacaggaaccagaagc

SEQ ID NO: 23 - DNA, 34 bp, Artificial Sequence
  Description of Artificial Sequence Synthetic primer
ggcaagtgacatgtccctataacttcgtataatg

SEQ ID NO: 24 - DNA, 26 bp, Artificial Sequence
  Description of Artificial Sequence Synthetic primer
ccctaagctcttcccttcttgttggc

SEQ ID NO: 25 - DNA, 27 bp, Artificial Sequence
  Description of Artificial Sequence Synthetic primer
ccttcagagccccacttcctgtcgaag

SEQ ID NO: 26 - DNA, 24 bp, Artificial Sequence
  Description of Artificial Sequence Synthetic primer
gaccacatgaagcagcacgacttc

SEQ ID NO: 27 - DNA, 29 bp, Artificial Sequence
  Description of Artificial Sequence Synthetic primer
gttacaacacctagaagtagtacctcctc

SEQ ID NO: 28 - DNA, 24 bp, Artificial Sequence
  Description of Artificial Sequence Synthetic primer
gtttagcaggctgtgcccttgccc

SEQ ID NO: 29 - DNA, 24 bp, Artificial Sequence
  Description of Artificial Sequence Synthetic primer
cgccaagcctgaatgaagtcctag

SEQ ID NO: 30 - DNA, 24 bp, Artificial Sequence
  Description of Artificial Sequence Synthetic primer
gaccacatgaagcagcacgacttc

SEQ ID NO: 31 - DNA, 24 bp, Artificial Sequence
  Description of Artificial Sequence Synthetic primer
cgccaagcctgaatgaagtcctag

SEQ ID NO: 32 - DNA, 26 bp, Artificial Sequence
  Description of Artificial Sequence Synthetic primer
ggacaggaagtgacactgggggtcag

SEQ ID NO: 33 - DNA, 23 bp, Artificial Sequence
  Description of Artificial Sequence Synthetic primer
ccagaaggaacccgagcttcatc

SEQ ID NO: 34 - DNA, 24 bp, Artificial Sequence
  Description of Artificial Sequence Synthetic primer
gaccacatgaagcagcacgacttc

SEQ ID NO: 35 - DNA, 23 bp, Artificial Sequence
  Description of Artificial Sequence Synthetic primer
ccagaaggaacccgagcttcatc